SEQUENCE LISTING (26 sequences)

SEQ ID NO: 1 - protein, 116 aa, Artificial Sequence
  Murine 4E11 VH region
DVQLQESGPGLVKPSQSLSLTCTVTGYSITSDYAWNWIRQFPGNKLEWMGYIGYNGRTSYNPSLKSRISITRDTSKNQFFLQLNYVTTEDTATFYCARLGRGFAYWGQGTLVTVSA

SEQ ID NO: 2 - protein, 107 aa, Artificial Sequence
  Murine 4E11 VL region
DILMTQSPSSMSVSLGDTVSITCHASQGINSNIGWLLQKPGKSFKGLIYHGTNLEDGVPSRFSGSGSGTDYSLTISSLESEDFADYYCVQYAQFPYTFGGGTKLEIK

SEQ ID NO: 3 - protein, 116 aa, Artificial Sequence
  Consensus humanized 4E11 VH region
  SITE (49)..(49): X at position 49 is I or M
  SITE (68)..(68): X at position 68 is V or I
  SITE (72)..(72): X at position 72 is V or R
QVQLQESGPGLVKPSQTLSLTCTVSGYSITSDYAWNWIRQPPGKGLEWXGYIGYNGRTSYNPSLKSRXTISXDTSKNQFSLKLSSVTAADTAVYYCARLGRGFAYWGQGTLVTVSS

SEQ ID NO: 4 - protein, 107 aa, Artificial Sequence
  Consensus humanized 4E11 VL region
  SITE (36)..(36): X at position 36 is Y or L
  SITE (44)..(44): X at position 44 is P or F
  SITE (46)..(46): X at position 46 is L or G
DIQMTQSPSSLSASVGDRVTITCHASQGINSNIGWXQQKPGKAXKXLIYHGTNLEDGVPSRFSGSGSGTDYTLTISSLQPEDFATYYCVQYAQFPYTFGQGTKLEIK

SEQ ID NO: 5 - protein, 116 aa, Artificial Sequence
  Humanized 4E11 VH region variant 1
QVQLQESGPGLVKPSQTLSLTCTVSGYSITSDYAWNWIRQPPGKGLEWIGYIGYNGRTSYNPSLKSRVTISVDTSKNQFSLKLSSVTAADTAVYYCARLGRGFAYWGQGTLVTVSS

SEQ ID NO: 6 - protein, 116 aa, Artificial Sequence
  Humanized 4E11 VH region variant 2
QVQLQESGPGLVKPSQTLSLTCTVSGYSITSDYAWNWIRQPPGKGLEWIGYIGYNGRTSYNPSLKSRVTISRDTSKNQFSLKLSSVTAADTAVYYCARLGRGFAYWGQGTLVTVSS

SEQ ID NO: 7 - protein, 116 aa, Artificial Sequence
  Humanized 4E11 VH region variant 3
QVQLQESGPGLVKPSQTLSLTCTVSGYSITSDYAWNWIRQPPGKGLEWMGYIGYNGRTSYNPSLKSRITISRDTSKNQFSLKLSSVTAADTAVYYCARLGRGFAYWGQGTLVTVSS

SEQ ID NO: 8 - protein, 107 aa, Artificial Sequence
  Humanized 4E11 VL region variant 1
DIQMTQSPSSLSASVGDRVTITCHASQGINSNIGWYQQKPGKAPKLLIYHGTNLEDGVPSRFSGSGSGTDYTLTISSLQPEDFATYYCVQYAQFPYTFGQGTKLEIK

SEQ ID NO: 9 - protein, 107 aa, Artificial Sequence
  Humanized 4E11 VL region variant 2
DIQMTQSPSSLSASVGDRVTITCHASQGINSNIGWLQQKPGKAPKGLIYHGTNLEDGVPSRFSGSGSGTDYTLTISSLQPEDFATYYCVQYAQFPYTFGQGTKLEIK

SEQ ID NO: 10 - protein, 107 aa, Artificial Sequence
  Humanized 4E11 VL region variant 3
DIQMTQSPSSLSASVGDRVTITCHASQGINSNIGWLQQKPGKAFKGLIYHGTNLEDGVPSRFSGSGSGTDYTLTISSLQPEDFATYYCVQYAQFPYTFGQGTKLEIK

SEQ ID NO: 11 - protein, 442 aa, Artificial Sequence
  Chimeric 4E11 heavy chain on human IgG4(S228P) background
DVQLQESGPGLVKPSQSLSLTCTVTGYSITSDYAWNWIRQFPGNKLEWMGYIGYNGRTSYNPSLKSRISITRDTSKNQFFLQLNYVTTEDTATFYCARLGRGFAYWGQGTLVTVSAASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 12 - protein, 214 aa, Artificial Sequence
  Chimeric 4E11 light chain on human kappa background
DILMTQSPSSMSVSLGDTVSITCHASQGINSNIGWLLQKPGKSFKGLIYHGTNLEDGVPSRFSGSGSGTDYSLTISSLESEDFADYYCVQYAQFPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 442 aa, Artificial Sequence
  Humanized 4E11 heavy chain variant 1 on human IgG4(S228P) background
QVQLQESGPGLVKPSQTLSLTCTVSGYSITSDYAWNWIRQPPGKGLEWIGYIGYNGRTSYNPSLKSRVTISVDTSKNQFSLKLSSVTAADTAVYYCARLGRGFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 14 - protein, 442 aa, Artificial Sequence
  Humanized 4E11 heavy chain variant 2 on human IgG4(S228P) background
QVQLQESGPGLVKPSQTLSLTCTVSGYSITSDYAWNWIRQPPGKGLEWIGYIGYNGRTSYNPSLKSRVTISRDTSKNQFSLKLSSVTAADTAVYYCARLGRGFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 15 - protein, 442 aa, Artificial Sequence
  Humanized 4E11 heavy chain variant 3 on human IgG4(S228P) background
QVQLQESGPGLVKPSQTLSLTCTVSGYSITSDYAWNWIRQPPGKGLEWMGYIGYNGRTSYNPSLKSRITISRDTSKNQFSLKLSSVTAADTAVYYCARLGRGFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 16 - protein, 214 aa, Artificial Sequence
  Humanized 4E11 light chain variant 1 on human kappa background
DIQMTQSPSSLSASVGDRVTITCHASQGINSNIGWYQQKPGKAPKLLIYHGTNLEDGVPSRFSGSGSGTDYTLTISSLQPEDFATYYCVQYAQFPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 214 aa, Artificial Sequence
  Humanized 4E11 light chain variant 2 on human kappa background
DIQMTQSPSSLSASVGDRVTITCHASQGINSNIGWLQQKPGKAPKGLIYHGTNLEDGVPSRFSGSGSGTDYTLTISSLQPEDFATYYCVQYAQFPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 214 aa, Artificial Sequence
  Humanized 4E11 light chain variant 3 on human kappa background
DIQMTQSPSSLSASVGDRVTITCHASQGINSNIGWLQQKPGKAFKGLIYHGTNLEDGVPSRFSGSGSGTDYTLTISSLQPEDFATYYCVQYAQFPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 19 aa, Artificial Sequence
  Signal peptide on heavy chain (exemplary)
MDWTWRILFLVAAATGTHA

SEQ ID NO: 20 - protein, 20 aa, Artificial Sequence
  Signal peptide on light chain (exemplary)
MVLQTQVFISLLLWISGAYG

SEQ ID NO: 21 - DNA, 1383 bp, Artificial Sequence
  DNA sequence of humanized 4E11 heavy chain variant 1 on human IgG4(S228P) background including an exemplary signal peptide
  MISC_FEATURE (1)..(57): Exemplary signal peptide sequence
atggactggacctggaggatcctgttcctggtggccgccgccaccggcacccacgcccaggtgcagctgcaggagtccggccccggcctggtgaagccctcccagaccctgtccctgacctgcaccgtgtccggctactccatcacctccgactacgcctggaactggatcaggcagccccccggcaagggcctggagtggatcggctacatcggctacaacggcaggacctcctacaacccctccctgaagtccagggtgaccatctccgtggacacctccaagaaccagttctccctgaagctgtcctccgtgaccgccgccgacaccgccgtgtactactgcgccaggctgggcaggggcttcgcctactggggccagggcaccctggtgaccgtgtcctccgcctccaccaagggcccctccgtgttccccctggccccctgctccaggtccacctccgagtccaccgccgccctgggctgcctggtgaaggactacttccccgagcccgtgaccgtgtcctggaactccggcgccctgacctccggcgtgcacaccttccccgccgtgctgcagtcctccggcctgtactccctgtcctccgtggtgaccgtgccctcctcctccctgggcaccaagacctacacctgcaacgtggaccacaagccctccaacaccaaggtggacaagagggtggagtccaagtacggccccccctgccccccctgccccgcccccgagttcctgggcggcccctccgtgttcctgttcccccccaagcccaaggacaccctgatgatctccaggacccccgaggtgacctgcgtggtggtggacgtgtcccaggaggaccccgaggtgcagttcaactggtacgtggacggcgtggaggtgcacaacgccaagaccaagcccagggaggagcagttcaactccacctacagggtggtgtccgtgctgaccgtgctgcaccaggactggctgaacggcaaggagtacaagtgcaaggtgtccaacaagggcctgccctcctccatcgagaagaccatctccaaggccaagggccagcccagggagccccaggtgtacaccctgcccccctcccaggaggagatgaccaagaaccaggtgtccctgacctgcctggtgaagggcttctacccctccgacatcgccgtggagtgggagtccaacggccagcccgagaacaactacaagaccaccccccccgtgctggactccgacggctccttcttcctgtactccaggctgaccgtggacaagtccaggtggcaggagggcaacgtgttctcctgctccgtgatgcacgaggccctgcacaaccactacacccagaagtccctgtccctgtccctgggc

SEQ ID NO: 22 - DNA, 1383 bp, Artificial Sequence
  DNA sequence of humanized 4E11 heavy chain variant 2 on human IgG4(S228P) background including an exemplary signal peptide
  MISC_FEATURE (1)..(57): Exemplary signal peptide sequence
atggactggacctggaggatcctgttcctggtggccgccgccaccggcacccacgcccaggtgcagctgcaggagtccggccccggcctggtgaagccctcccagaccctgtccctgacctgcaccgtgtccggctactccatcacctccgactacgcctggaactggatcaggcagccccccggcaagggcctggagtggatcggctacatcggctacaacggcaggacctcctacaacccctccctgaagtccagggtgaccatctccagggacacctccaagaaccagttctccctgaagctgtcctccgtgaccgccgccgacaccgccgtgtactactgcgccaggctgggcaggggcttcgcctactggggccagggcaccctggtgaccgtgtcctccgcctccaccaagggcccctccgtgttccccctggccccctgctccaggtccacctccgagtccaccgccgccctgggctgcctggtgaaggactacttccccgagcccgtgaccgtgtcctggaactccggcgccctgacctccggcgtgcacaccttccccgccgtgctgcagtcctccggcctgtactccctgtcctccgtggtgaccgtgccctcctcctccctgggcaccaagacctacacctgcaacgtggaccacaagccctccaacaccaaggtggacaagagggtggagtccaagtacggccccccctgccccccctgccccgcccccgagttcctgggcggcccctccgtgttcctgttcccccccaagcccaaggacaccctgatgatctccaggacccccgaggtgacctgcgtggtggtggacgtgtcccaggaggaccccgaggtgcagttcaactggtacgtggacggcgtggaggtgcacaacgccaagaccaagcccagggaggagcagttcaactccacctacagggtggtgtccgtgctgaccgtgctgcaccaggactggctgaacggcaaggagtacaagtgcaaggtgtccaacaagggcctgccctcctccatcgagaagaccatctccaaggccaagggccagcccagggagccccaggtgtacaccctgcccccctcccaggaggagatgaccaagaaccaggtgtccctgacctgcctggtgaagggcttctacccctccgacatcgccgtggagtgggagtccaacggccagcccgagaacaactacaagaccaccccccccgtgctggactccgacggctccttcttcctgtactccaggctgaccgtggacaagtccaggtggcaggagggcaacgtgttctcctgctccgtgatgcacgaggccctgcacaaccactacacccagaagtccctgtccctgtccctgggc

SEQ ID NO: 23 - DNA, 1383 bp, Artificial Sequence
  DNA sequence of humanized 4E11 heavy chain variant 3 on human IgG4(S228P) background including an exemplary signal peptide
  MISC_FEATURE (1)..(57): Exemplary signal peptide sequence
atggactggacctggaggatcctgttcctggtggccgccgccaccggcacccacgcccaggtgcagctgcaggagtccggccccggcctggtgaagccctcccagaccctgtccctgacctgcaccgtgtccggctactccatcacctccgactacgcctggaactggatcaggcagccccccggcaagggcctggagtggatgggctacatcggctacaacggcaggacctcctacaacccctccctgaagtccaggatcaccatctccagggacacctccaagaaccagttctccctgaagctgtcctccgtgaccgccgccgacaccgccgtgtactactgcgccaggctgggcaggggcttcgcctactggggccagggcaccctggtgaccgtgtcctccgcctccaccaagggcccctccgtgttccccctggccccctgctccaggtccacctccgagtccaccgccgccctgggctgcctggtgaaggactacttccccgagcccgtgaccgtgtcctggaactccggcgccctgacctccggcgtgcacaccttccccgccgtgctgcagtcctccggcctgtactccctgtcctccgtggtgaccgtgccctcctcctccctgggcaccaagacctacacctgcaacgtggaccacaagccctccaacaccaaggtggacaagagggtggagtccaagtacggccccccctgccccccctgccccgcccccgagttcctgggcggcccctccgtgttcctgttcccccccaagcccaaggacaccctgatgatctccaggacccccgaggtgacctgcgtggtggtggacgtgtcccaggaggaccccgaggtgcagttcaactggtacgtggacggcgtggaggtgcacaacgccaagaccaagcccagggaggagcagttcaactccacctacagggtggtgtccgtgctgaccgtgctgcaccaggactggctgaacggcaaggagtacaagtgcaaggtgtccaacaagggcctgccctcctccatcgagaagaccatctccaaggccaagggccagcccagggagccccaggtgtacaccctgcccccctcccaggaggagatgaccaagaaccaggtgtccctgacctgcctggtgaagggcttctacccctccgacatcgccgtggagtgggagtccaacggccagcccgagaacaactacaagaccaccccccccgtgctggactccgacggctccttcttcctgtactccaggctgaccgtggacaagtccaggtggcaggagggcaacgtgttctcctgctccgtgatgcacgaggccctgcacaaccactacacccagaagtccctgtccctgtccctgggc

SEQ ID NO: 24 - DNA, 702 bp, Artificial Sequence
  DNA sequence of humanized 4E11 light chain variant 1 on human kappa background including an exemplary signal peptide
  MISC_FEATURE (1)..(60): Exemplary signal peptide sequence
atggtgctgcagacccaggtgttcatctccctgctgctgtggatctccggcgcctacggcgacatccagatgacccagtccccctcctccctgtccgcctccgtgggcgacagggtgaccatcacctgccacgcctcccagggcatcaactccaacatcggctggtaccagcagaagcccggcaaggcccccaagctgctgatctaccacggcaccaacctggaggacggcgtgccctccaggttctccggctccggctccggcaccgactacaccctgaccatctcctccctgcagcccgaggacttcgccacctactactgcgtgcagtacgcccagttcccctacaccttcggccagggcaccaagctggagatcaagaggaccgtggccgccccctccgtgttcatcttccccccctccgacgagcagctgaagtccggcaccgcctccgtggtgtgcctgctgaacaacttctaccccagggaggccaaggtgcagtggaaggtggacaacgccctgcagtccggcaactcccaggagtccgtgaccgagcaggactccaaggactccacctactccctgtcctccaccctgaccctgtccaaggccgactacgagaagcacaaggtgtacgcctgcgaggtgacccaccagggcctgtcctcccccgtgaccaagtccttcaacaggggcgagtgc

SEQ ID NO: 25 - DNA, 702 bp, Artificial Sequence
  DNA sequence of humanized 4E11 light chain variant 2 on human kappa background including an exemplary signal peptide
  MISC_FEATURE (1)..(60): Exemplary signal peptide sequence
atggtgctgcagacccaggtgttcatctccctgctgctgtggatctccggcgcctacggcgacatccagatgacccagtccccctcctccctgtccgcctccgtgggcgacagggtgaccatcacctgccacgcctcccagggcatcaactccaacatcggctggctgcagcagaagcccggcaaggcccccaagggcctgatctaccacggcaccaacctggaggacggcgtgccctccaggttctccggctccggctccggcaccgactacaccctgaccatctcctccctgcagcccgaggacttcgccacctactactgcgtgcagtacgcccagttcccctacaccttcggccagggcaccaagctggagatcaagaggaccgtggccgccccctccgtgttcatcttccccccctccgacgagcagctgaagtccggcaccgcctccgtggtgtgcctgctgaacaacttctaccccagggaggccaaggtgcagtggaaggtggacaacgccctgcagtccggcaactcccaggagtccgtgaccgagcaggactccaaggactccacctactccctgtcctccaccctgaccctgtccaaggccgactacgagaagcacaaggtgtacgcctgcgaggtgacccaccagggcctgtcctcccccgtgaccaagtccttcaacaggggcgagtgc

SEQ ID NO: 26 - DNA, 702 bp, Artificial Sequence
  DNA sequence of humanized 4E11 light chain variant 3 on human kappa background including an exemplary signal peptide
  MISC_FEATURE (1)..(60): Exemplary signal peptide sequence
atggtgctgcagacccaggtgttcatctccctgctgctgtggatctccggcgcctacggcgacatccagatgacccagtccccctcctccctgtccgcctccgtgggcgacagggtgaccatcacctgccacgcctcccagggcatcaactccaacatcggctggctgcagcagaagcccggcaaggccttcaagggcctgatctaccacggcaccaacctggaggacggcgtgccctccaggttctccggctccggctccggcaccgactacaccctgaccatctcctccctgcagcccgaggacttcgccacctactactgcgtgcagtacgcccagttcccctacaccttcggccagggcaccaagctggagatcaagaggaccgtggccgccccctccgtgttcatcttccccccctccgacgagcagctgaagtccggcaccgcctccgtggtgtgcctgctgaacaacttctaccccagggaggccaaggtgcagtggaaggtggacaacgccctgcagtccggcaactcccaggagtccgtgaccgagcaggactccaaggactccacctactccctgtcctccaccctgaccctgtccaaggccgactacgagaagcacaaggtgtacgcctgcgaggtgacccaccagggcctgtcctcccccgtgaccaagtccttcaacaggggcgagtgc

(19) United States
(12) Patent Application Publication
(10) Pub. No.: US 2024/0400697 A1
(43) Pub. Date: Dec. 5, 2024

(54) HUMANIZED ANTI-EGFR VIII ANTIBODIES AND ANTIGEN-BINDING FRAGMENTS THEREOF

(71) Applicant: NATIONAL RESEARCH COUNCIL OF CANADA, Ottawa (CA)

(72) Inventors: Anne MARCIL, Pierrefonds, Québec (CA); Maria JARAMILLO, Beaconsfield, Québec (CA); Traian SULEA, Kirkland, Québec (CA); Maria MORENO, Ottawa, Ontario (CA); Cunle WU, Montreal, Québec (CA)

(21) Appl. No.: 18/696,661

(22) PCT Filed: Sep. 29, 2021
(86) PCT No.: PCT/IB2021/058954

§ 371 Date: Mar. 28, 2024

(51) Int. Cl. C07K 16/28; A61K 35/17; A61K 39/00; A61K 47/68; G01N 33/53; G01N 33/68

(52) U.S. Cl. CPC C07K 16/2863; A61K 35/17; A61K 39/4611; A61K 39/4613; A61K 39/464404; A61K 47/6845; G01N 33/53; G01N 33/68; C07K 2317/24; C07K 2317/52; C07K 2317/565; C07K 2317/92; C07K 2317/94; G01N 2333/71

(57) ABSTRACT

Antigen-binding agents such as humanized antibodies or antigen-binding fragments thereof, that specifically bind to epidermal growth factor receptor variant III (EGFRvIII) are provided. The EGFRVIII-specific humanized antibodies or antigen-binding fragments thereof may be used for the treatment of cancer.

### TECHNICAL FIELD

The present disclosure provides humanized antigen-binding agents such as antibodies or antigen-binding fragments thereof, that specifically bind to epidermal growth factor receptor variant III (EGFRvIII). The EGFRvIII-specific antibodies or antigen-binding fragments thereof, of the instant disclosure may be used, for example, for the treatment of cancer, as antibody-drug conjugates, radio-immuno-conjugates, chimeric antigen receptors (CARs) and bispecific T-cell engagers (BiTEs) targeting EGFRvIII-expressing cells.

### BACKGROUND

The epidermal growth factor receptor variant III (EGFRvIII) is amplified, highly expressed and present in 25-64% of glioblastoma multiforme (GBM). It should be noted that the different detection methods yielded inconsistent results, but EGFRvIII mRNA and protein expression has been detected in a subset of carcinomas of the breast as well as in head and neck squamous cell carcinoma (HNSCC) using multiple complementary techniques (reviewed in Gan et al 2013). Unlike wild type (wt) epidermal growth factor receptor (EGFR), which is expressed in tissues of epithelial, mesenchymal and neuronal origin and plays a major role in normal cellular processes such as proliferation, differentiation and development, EGFRvIII is not expressed on normal tissues.

The EGFRvIII variant originates from an in-frame deletion of exons 2-7 of the EGFR gene resulting in the removal of a sequence encoding 267 amino acid residues of the extracellular domain. The newly formed splice junction encodes a glycine residue which has no counterpart in wild type human EGFR and therefore forms a neo-epitope. Moreover, numerous studies showed that normal tissues are devoid of EGFRvIII. EGFRvIII thus contains a new tumor specific cell surface epitope that could be exploited for antibody targeted therapies. However, the EGFRvIII neo-epitope is not very immunogenic compared to the remaining of the human sequence, and many of the antibodies generated to date have not been shown to be specifically recognizing EGFRvIII (reviewed in Gan et al 2013).

In rare cases, monoclonal antibodies (mAbs) directed against the EGFRvIII neo-epitope have been described, including antibody 13.1.2 (U.S. Pat. No. 7,736,644) which is also being developed as an antibody drug conjugate (ADC) by Amgen (AMG 595: Hamblett K. J, et al., Molecular Cancer Therapeutics, Vol. 14 (7), pp. 1614-24, 2015). U.S. Pat. No. 9,562,102 also describes the 806 antibody developed by Abbvie (ABT-806, ABT-414), which binds to EGFRvIII as well as a subset of amplified EGFR on EGFR overexpressing tumor cells (Cleary, J M et al., Invest New Drugs, 33 (3), pp. 671-8, 2015; Reilly, E B., Molecular Cancer Therapeutics, Vol. 14 (5), pp. 1141-51, 2015). Although this antibody has been shown to bind preferentially to tumor EGFR in preclinical models, binding of this antibody to wt EGFR present in human skin has been shown to account for the cutaneous toxicity that ABT-806 exhibits in some patients (Cleary et al 2015).

Humanized antibodies or antigen-binding fragments thereof that specifically target an epitope of EGFRvIII that is absent or not accessible in EGFR-expressing cells would be beneficial for improved therapeutics (i.e. cancer therapeutics).

### SUMMARY

Antigen-binding agents such as humanized antibodies or humanized antigen-binding fragments thereof, which specifically bind to epidermal growth factor receptor variant III (EGFRvIII) are provided.

As described in more details below, some anti-EGFRvIII humanized antibodies or antigen-binding fragments thereof may bind to EGFRvIII at the surface of cancer cells (e.g., glioblastoma cells). In some embodiments, the humanized antibodies or antigen-binding fragments thereof do not significantly bind to EGFR expressed on cancer cells (e.g., U87MG-EGFR WT).

The humanized antibodies or antigen-binding fragments thereof of the present disclosure may be internalized by cancer cells and may thus be used, in an aspect thereof, for delivery of cargo molecules. Particularly contemplated are anti-EGFRvIII humanized antibodies or antigen-binding fragments thereof that are conjugated to therapeutic moieties. The anti-EGFRvIII humanized antibodies described herein may be used for inhibiting the growth of EGFRvIII-expressing tumor cells.

In some embodiments of the present disclosure, the anti-EGFRvIII humanized antibodies or antigen-binding fragments thereof may be able to bind to an epitope present in both native EGFRvIII (e.g., native recombinant EGFRvIII) and denatured EGFRvIII (e.g., denatured recombinant EGFRvIII).

Embodiments of the disclosure particularly include anti-EGFRvIII humanized antibodies or antigen-binding fragments thereof comprising the complementarity determining region (CDR) of the anti-EGFRvIII 4E11 antibody.

In some embodiments, an antigen-binding agent, antigen-binding domain, antibody or antigen-binding fragment thereof that specifically binds to epidermal growth factor receptor variant III (EGFRvIII), wherein the antigen-binding agent, antigen-binding domain, antibody or antigen-binding fragment comprises:
a heavy chain variable region comprising the amino acid sequence QVQLQESGPGLVKPSQTLSLTCTVSGYSITSDYAWNWIRQPPGKGLEWX₁GYI GYNGRTSYNPSLKSRX₂TISX₃DTSKNQFSLKLSSVTAADTAVYYCARLGRGFAY WGQGTLVTVSS (SEQ ID NO:3), where X1=I or M, X2=V or I, and X3=V or R; and,
a light chain variable region comprising the amino acid sequence DIQMTQSPSSLSASVGDRVTITCHASQGINSNIGWX₄QQKPGKAX₅KXGLIYHGT NLEDGVPSRFSGSGSGTDYTLTISSLOPEDFATYYCVQYAQFPYTFGQGTKLEI K (SEQ ID NO:4), where X₄=Y or L, X₅=P or F, and X₆=L or G.

In some embodiments, the antigen-binding agent, antigen-binding domain, antibody or antigen-binding fragment comprises:
the heavy chain variable sequence is selected from any one of SEQ ID NO: 5, SEQ ID NO: 6 and SEQ ID NO 7; and,
the light chain variable sequence is selected from any one of SEQ ID NO: 8, SEQ ID NO: 9 and SEQ ID NO: 10.

In an embodiment, the antigen-binding agent, antigen-binding domain, antibody or antigen-binding fragment is a chimeric antigen receptor, a bi-specific T-cell engager, a bispecific killer cell engager, a trispecific killer cell engager or any immunotherapeutic compound, such as an antibody drug conjugate (ADC) or a compound used for detection, such as a radioimmunoconjugate.

In an embodiment, the antigen-binding agent, antigen-binding domain, antibody or antigen-binding fragment is a monoclonal antibody, a polyclonal antibody, a humanized antibody, a chimeric antibody, a human antibody, a single chain antibody, or a multispecific antibody. In an embodiment, the antibody or antigen-binding fragment thereof comprises a human IgG constant region. In an embodiment, antibody or antigen-binding fragment thereof comprises a human IgG4 constant region.

In an embodiment, the antigen-binding agent, antigen-binding domain, antibody or antigen-binding fragment comprises a human IgG4 constant region bearing the S228P mutation.

In an embodiment, the antigen-binding agent, antigen-binding domain, antibody or antigen-binding fragment specifically binds to EGFRvIII wherein the antigen-binding agent, antigen-binding domain, antibody or antigen-binding fragment comprises a heavy chain sequence and a light chain sequence, wherein:
the heavy chain sequence is SEQ ID NO: 13, SEQ ID NO: 14 or SEQ ID NO: 15; and,
the light chain sequence is SEQ ID NO: 16, SEQ ID NO: 17 or SEQ ID NO: 18.

In an embodiment, the antigen-binding agent, antigen-binding domain, antibody or antigen-binding comprises a scFv, a Fab, a Fab′ or a (Fab′)₂.

In an embodiment, the antigen-binding agent, antigen-binding domain, antibody or antigen-binding fragment is linked to a cargo molecule. In some embodiments, the cargo molecule comprises a therapeutic moiety. In some embodiments, the therapeutic moiety comprises a cytotoxic agent, a cytostatic agent, an anti-cancer agent or a radiotherapeutic. In some embodiments, the antigen-binding agent, antigen-binding domain, antibody or antigen-binding fragment is conjugated to a detectable moiety.

In an embodiment, there is provide a pharmaceutical composition comprising the antigen-binding agent, antigen-binding domain, antibody or antigen-binding fragment of the present invention and a pharmaceutically acceptable carrier, diluent or excipient.

In an embodiment, there is provided a nucleic acid molecule encoding a heavy chain variable region and/or a light chain variable region of the antigen-binding agent, antigen-binding domain, antibody or antigen-binding fragment of the present invention.

In an embodiment, there is provided a kit comprising at least one of the antigen-binding agent, antigen-binding domain, antibody or antigen-binding fragment of the present invention.

In an embodiment, there is provided a vector or set of vectors comprising a nucleic acid sequence encoding a heavy chain variable region and a light chain variable region of the antigen-binding agent, antigen-binding domain, antibody or antigen-binding fragment of the present invention. In an embodiment, there is provided an isolated cell comprising the vector or set of vectors. In some embodiments, the isolated cell is capable of expressing, assembling and/or secreting an antigen-binding agent, antigen-binding domain, antibody or antigen-binding fragment thereof.

There is also provided a kit comprising a first vial comprising a nucleotide or vector encoding the light chain of the antigen-binding agent, antigen-binding domain, antibody or antigen-binding fragment of the present invention and a second vial comprising a nucleotide or vector encoding the heavy chain of the antigen-binding agent, antigen-binding domain, antibody or antigen-binding fragment of the present invention.

There is also provided a method of treating cancer comprising cells expressing EGFRvIII, the method comprising administering the antigen-binding agent, antigen-binding domain, antibody or antigen-binding fragment of the present invention to a subject in need. In an embodiment, the antigen-binding agent, antigen-binding domain, antibody or antigen-binding fragment is used in combination with a chemotherapeutic. In an embodiment, the subject in need has or is suspected of having gliobastoma multiforme, has or is suspected of having a carcinoma, breast carcinoma or HNSCC.

There is also provided a method of detecting EGFRvIII, the method comprising contacting a sample comprising or suspected of comprising EGFRvIII with the antigen-binding agent, antigen-binding domain, antibody or antigen-binding fragment of any one of the present invention.

In an embodiment, the method of making the antigen-binding agent, antigen-binding domain, antibody or antigen-binding fragment comprises culturing a cell comprising nucleic acids encoding said antigen-binding agent, antigen-binding domain, antibody or antigen-binding fragment so that the antigen-binding agent, antigen-binding domain, antibody or antigen-binding fragment is produced. In an embodiment, the method provided further comprises conjugating the antigen-binding agent, antigen-binding domain, antibody or antigen-binding fragment thereof with a cargo molecule. In an embodiment, the cargo molecule comprises a therapeutic or detectable moiety.

There is also provided a method of treating subject having a cancer associated with EGFRvIII expression, the method comprising administering cells expressing the antigen-binding agent, antigen-binding domain, antibody or antigen-binding fragment of the present invention, wherein the antigen-binding agent, antigen-binding domain, antibody or antigen-binding fragment is a chimeric antigen receptor, a bi-specific T-cell engager, a bispecific killer cell engager or a trispecific killer cell engage or an antibody drug conjugate. In an embodiment, the subject in need has or is suspected of having glioma, gliobastoma multiforme. Carcinoma, breast carcinoma, oral carcinoma or HNSCC. In an embodiment, the method provided comprises cells that are T-cells, NK cells or immune cells autologous to the subject. In an embodiment, the isolated cell population is engineered to express the antigen-binding agent, antigen-binding domain, antibody or antigen-binding fragment of the present invention. In an embodiment, the isolated cell population is of human origin. In an embodiment, the isolated cell population comprises T cells, Natural Killer (NK) cells, cytotoxic T cells, regulatory T cells, and combinations thereof. In an embodiment, the T-cells comprise CD4+ T-cells, CD8+ T-cells or a combination thereof.

In an embodiment, the isolated cell population is engineered to express another chimeric antigen receptor having affinity for another antigen of the same target or of a different target. In an embodiment, the isolated cell population comprises a host's immune cells.

There is provided a pharmaceutical composition comprising the isolated cell population and a pharmaceutically acceptable buffer or excipient.

In accordance with the present disclosure, the humanized antibody or antigen-binding fragment thereof may have, for example, an affinity of less than 100 nM such as for example, an affinity of 50 nM or less, 20 nM or less, 10 nM or less, 5 nM or less, etc.

Embodiments of the present disclosure include humanized antibodies or antigen-binding fragments thereof which may comprise a human IgG constant region. Humanized antibodies or antigen-binding fragments of the present disclosure may comprise, for example and without limitation, a human IgG1 constant region, human IgG2 constant region or human IgG4 constant region.

In accordance with the present disclosure, the humanized antibody or antigen-binding fragment thereof may be a full-size IgG antibody, a single chain antibody, or a multispecific antibody (e.g., a bispecific antibody).

Bispecific antibodies or antigen-binding fragments thereof of the present disclosure includes those that may comprise a first arm that specifically binds to a first human EGFRvIII epitope and a second arm that specifically binds to a second (non-overlapping) human EGFRvIII epitope (e.g. a biparatopic antibody).

Additional embodiments of bispecific antibodies or antigen-binding fragments thereof of the present disclosure includes those that may comprise a first arm that specifically binds to a first human EGFRvIII epitope and a second arm that specifically binds to another antigen.

The bispecific antibody or antigen-binding fragment thereof of the present disclosure include bispecific immune cell engagers such as those comprising a first arm that specifically binds to human EGFRvIII and a second arm that specifically binds to CD3.

In accordance with the present disclosure, the antigen-binding fragment comprises, for example, a scFv, a Fab, a Fab′ or a (Fab′)₂.

In a further aspect, the present disclosure provides anti-EGFRvIII humanized antibodies or antigen-binding fragments thereof which may be linked to a cargo molecule.

In accordance with the present disclosure, the cargo molecule may comprise a therapeutic moiety, such as for example, a cytotoxic agent, a cytostatic agent, an anti-cancer agent or a radiotherapeutic. In particular embodiments of the disclosure, the antibody drug conjugates may comprise a cytotoxic agent. Another particular embodiment of the disclosure relates to antibody drug conjugates comprising a radiotherapeutic.

In accordance with the present disclosure, the cargo molecule may comprise a detectable moiety.

The humanized antibodies or antigen-binding fragments thereof of the present disclosure may be provided in the form of pharmaceutical compositions. The pharmaceutical composition may comprise, for example, a pharmaceutically acceptable carrier, diluent or excipient.

The present disclosure additionally provides nucleic acid molecules encoding a light chain variable region and/or a heavy chain variable region of the humanized antibody or antigen-binding fragment thereof disclosed herein.

In a further aspect, the present disclosure provides a kit comprising at least one of the humanized antibody or antigen-binding fragments thereof disclosed herein.

Additional aspects of the present disclosure relate to a vector or set of vectors which may comprise a nucleic acid encoding a light chain variable region and a heavy chain variable region of the humanized antibody or antigen-binding fragment disclosed herein. The nucleic acids encoding the light chain variable region or the light chain and the heavy chain variable region or the heavy chain may be provided on the same vector or on separate vectors.

Further aspects of the present disclosure relate to isolated cells comprising the vector or set of vectors described herein. The isolated cells may be capable of expressing, assembling and/or secreting the humanized antibody or antigen-binding fragment thereof.

Other aspects of the present disclosure relate to a kit comprising a first vial comprising a nucleic acid or vector encoding the light chain of the humanized antibody or antigen-binding fragment thereof of the present disclosure and a second vial comprising a nucleic acid or vector encoding the heavy chain of the humanized antibody or antigen-binding fragment thereof.

Additional aspects of the present disclosure relate to a method of treating cancer which comprises cells (e.g., tumor cells) expressing EGFRvIII. The method may comprise administering the humanized antibody or antigen-binding fragment thereof described herein to subject in need. Antibody or antigen-binding fragments that are conjugated to a therapeutic moiety (ADCs) are particularly contemplated in methods of treatments.

The present disclosure additionally relates to the use of the humanized antibody or antigen-binding fragment thereof described herein in the treatment of cancer.

The present disclosure further relates to the use of the humanized antibody or antigen-binding fragment thereof described herein in the manufacture of a medicament for the treatment of cancer.

In accordance with the present disclosure, the humanized antibody or antigen-binding fragment thereof may be used in combination with a chemotherapeutic.

In accordance with the present disclosure, the subject in need has or is suspected of having gliobastoma multiforme.

Further in accordance with the present disclosure, the subject in need has or is suspected of having a carcinoma.

Further aspects of the present disclosure relate to a method of detecting EGFRvIII. The method may comprise contacting a sample comprising or suspected of comprising EGFRvIII with the humanized antibody or antigen-binding fragment described herein.

Additional aspects of the present disclosure relate to a method of making the humanized antibody or antigen-binding fragment thereof of the present disclosure by culturing a cell comprising nucleic acids or vectors encoding the humanized antibody or antigen-binding fragment so that the humanized antibody or antigen-binding fragment thereof is produced. The humanized antibody or antigen-binding fragment thereof may thus be isolated and/or purified.

The method may further comprise conjugating the humanized antibody or antigen-binding fragment thereof with a cargo molecule, such as for example, a therapeutic moiety.

Further scope, applicability and advantages of the present disclosure will become apparent from the non-restrictive detailed description given hereinafter. It should be understood, however, that this detailed description, while indicating exemplary embodiments of the disclosure, is given by way of example only, with reference to the accompanying drawings.

### BRIEF DESCRIPTION OF THE DRAWINGS

FIG. 1 provides sequence alignments of the selected human germline V-region and J-region sequences from the VBASE2 database (Retter et al, 2005) used for humanization of the murine 4E11 heavy chain variable region (mVH) sequence (SEQ ID NO:1) and of the murine 4E11 light chain variable region (mVL) sequence (SEQ ID NO:2).

FIG. 2 provides a 3D model of the variable region of murine 4E11 antibody. The heavy chain is colored in black and the light chain is colored in gray. The CDR is highlighted by a molecular surface mesh, and CDR loops are labeled. Arrows point to ball-and-stick models that highlight the 6 positions in the framework region that were selected for back-mutations in some of the humanized variants (i.e., amino-acid type retained as in the murine sequence, as labeled).

FIGS. 3A-B provides sequence alignments between the humanized heavy chain variable regions (SEQ ID NOS: 5, 6 and 7) of the 4E11 antibody (FIG. 3A) and between the humanized light chain variable regions (SEQ ID NOS: 8, 9 and 10) of the 4E11 antibody (FIG. 3B). The humanized heavy chain variable regions are referred to in FIG. 3A, and herein, as hVH1, hVH2 and hVH3. CDR loops are delineated according to the Kabat definition except for the CDR-H1 which is delineated by combining the Kabat and Chothia definitions. The humanized light chain variable regions are referred to in FIG. 3B, and herein, as hVL1, hVL2 and hVL3. For some of the humanized sequences, back-mutations to the parental amino-acid residues found at these positions in the murine 4E11 antibody are highlighted as white letters on black background. Back-mutated positions are labeled and indicated by arrows above the sequence alignment.

FIG. 4 shows overlaid thermograms determined by differential scanning calorimetry for the chimeric 4E11 antibody (cH-cL) and the 9 humanized variants of the present disclosure. See Table 1 for the associated melting temperatures (Tₘ) derived from integration of the individual thermograms for each variant. The humanized light chain variants are referred to herein as hL1, hL2 and hL3. The humanized heavy chain variants are referred to herein as hH1, hH2 and hH3. For clarity, hVH1, hVH2, hVH3 refer to the variable regions on the heavy chain and hVL1, hVL2, hVL3 refer to the variable regions on the light chain. While hH1, hH2 and hH3 refer to the heavy chain comprising the respective heavy chain variable region. For example, hH1 comprises hVH1 and the constant regions of the heavy chain, for example hH1=hVH1+CH1+CH2+CH3. Similarly, hVL1, hVL2, hVL3 refer to the variable regions on the light chain, while hL1, hL2 and hL3 refer to the light chain comprising the respective light chain variable region. For example, hL1 comprises hVL1 and the constant region of the light chain, for example hL1=hVL1+light chain constant region.

FIG. 5 shows dose-response binding curves obtained from flow cytometry data of purified humanized and chimeric 4E11 antibodies on U87MG glioblastoma cell lines overexpressing EGFR vIII (U87MG EGFR vIII).

FIG. 6 shows dose-response binding curves obtained from flow cytometry data of select purified humanized and chimeric 4E11 antibodies on U87MG glioblastoma cell lines overexpressing wild-type EGFR (U87MG EGFR wt).

### DETAILED DESCRIPTION

As used herein the term “EGFRvIII” refers to epidermal growth factor receptor variant III. The terms “EGFRvIII” and “vIII” are used interchangeably.

As used herein the term “EGFR” refers to human epidermal growth factor receptor. The term “wt EGFR”, “WT EGFR”, “EGFR WT” or “EGFR wt” are used interchangeably and refers to wild type EGFR.

The use of the terms “a” and “an” and “the” and similar referents in the context of describing the disclosure (especially in the context of the claims) are to be construed to cover both the singular and the plural, unless otherwise indicated herein or clearly contradicted by context.

Unless specifically stated or obvious from context, as used herein the term “or” is understood to be inclusive and covers both “or” and “and”.

The term “and/or” where used herein is to be taken as specific disclosure of each of the specified features or components with or without the other.

The terms “comprising”, “having”, “including”, and “containing” are to be construed as open-ended terms (i.e., meaning “including, but not limited to”) unless otherwise noted. The term “consisting of” is to be construed as close-ended.

As used herein the term “native” with respect to a protein such as EGFRvIII or EGFR refers to the natural conformation of the protein and includes proteins that are properly folded and/or functional.

As used herein the term “denatured” with respect to a protein such EGFRvIII or EGFR refers to a protein that has lost its natural conformation and may entail for example, a loss in the tertiary and secondary structure.

As used herein the expression “a peptide comprising or consisting of an EGFRvIII fragment” means that the peptide may comprise a portion other than the EGFRvIII fragment or that it consists in the EGFRvIII fragment.

As used herein, the term “bind” or “binding” of a targeting moiety means an at least temporary interaction or association with or to a target molecule, e.g., to human EGFRvIII and/or a mutational variant of EGFRvIII, e.g., as described herein.

As used herein the term “binds to an epitope comprising amino acid residues” means that the amino acid residue is either part of the epitope or that it is necessary for binding of the antibody.

As used herein the term “fails to bind to” a peptide or protein means that the antibody or antigen binding fragment a) does not bind significantly to the peptide or protein when expressed recombinantly or in cells, b) no detectable binding is observed, c) has similar binding property as a negative control antibody, d) does not binds specifically or e) binds with a value between 0% and 15% as determined by the flow cytometry experiments.

As used herein the term “autologous” refers to material derived from the same individual.

As used herein the term “antigen-binding domain” refers to the domain of an antibody or of an antigen-binding fragment which allows specific binding to an antigen.

As used herein, the term “antibody” encompasses monoclonal antibody, polyclonal antibody, humanized antibody, chimeric antibody, human antibody, single domain antibody (such as a VHH, VH, VL, nanobody, or any camelid or llama single domain antibody), multispecific antibody (e.g., bispecific antibodies) etc. The term “antibody” encompasses molecules that have a format similar to those occurring in nature (e.g., human IgGs, etc.). The term “antibody”, also referred to in the art as “immunoglobulin” (Ig), as used herein refers to a protein constructed from paired heavy and light polypeptide chains; various Ig isotypes exist, including IgA, IgD, IgE, IgG, and IgM. When an antibody is correctly folded, each chain folds into a number of distinct globular domains joined by more linear polypeptide sequences. For example, the immunoglobulin light chain folds into a variable (VL) and a constant (CL) domain, while the heavy chain folds into a variable (VH) and three constant (CH1, CH2, CH3) domains. Interaction of the heavy and light chain variable domains (VH and VL) results in the formation of an antigen-binding region (Fv). Each domain has a well-established structure familiar to those of skill in the art.

Typically, an antibody is constituted from the pairing of two light chains and two heavy chains. Different antibody isotypes exist, including IgA, IgD, IgE, IgG and IgM. Human IgGs are further divided into four distinct sub-groups namely; IgG1, IgG2, IgG3 and IgG4. Therapeutic antibodies are generally developed as IgG1, IgG2 or IgG4.

The antibody or antigen-binding fragment of the present disclosure may comprise, for example, a human IgG4 constant region or a fragment thereof. In exemplary embodiments, the antibody or antigen-binding fragment of the present disclosure may comprise, for example, a human IgG4 constant region carrying the S228P mutation or a fragment thereof. Constant regions of other antibody subtypes including human IgG1 and human IgG2 as well as other isotypes are also contemplated.

The light chain and heavy chain of human antibody IgG isotypes each comprise a variable region having 3 hypervariable regions named complementarity determining regions (CDRs). The light chain CDRs are identified herein as CDRL1, CDRL2 and CDRL3. The heavy chain CDRs are identified herein as CDRH1, CDRH2 and CDRH3. Complementarity determining regions are flanked by framework regions (FR) in the order: FR1-CDR1-FR2-CDR2-FR3-CDR3-FR4. The light and heavy chain variable regions are responsible for binding the target antigen and can therefore show significant sequence diversity between antibodies. The constant regions show less sequence diversity and are responsible for binding a number of natural proteins to elicit important biochemical events. The variable region of an antibody contains the antigen-binding determinants of the molecule, and thus determines the specificity of an antibody for its target antigen. The majority of sequence variability occurs in the CDRs which combine to form the antigen-binding site and contribute to binding and recognition of an antigenic determinant. The framework regions may play a role in the proper positioning and alignment in three dimensions of the CDRs for optimal antigen-binding. The specificity and affinity of an antibody for its antigen is determined by the structure of the hypervariable regions, as well as their size, shape, and chemistry of the surface they present to the antigen. Various schemes exist for identification of the regions of hypervariability, the two most common being those of Kabat and of Chothia and Lesk. Kabat et al (1991) define the “complementarity-determining regions” (CDR) based on sequence variability at the antigen-binding regions of the VH and VL domains. Chothia and Lesk (1987) define the “hypervariable loops” (H or L) based on the location of the structural loop regions in the VH and VL domains. These individual schemes define CDR and hypervariable loop regions that are adjacent or overlapping, those of skill in the antibody art often utilize the terms “CDR” and “hypervariable loop” interchangeably, and they may be so used herein. The CDR/loops are identified herein according to the Kabat scheme, except the CDRH1 loops that is delineated by combining the Kabat and Chothia definitions.

As used herein, “substantial identity” or “substantially identical” is meant a polypeptide sequence that has the same polypeptide sequence, respectively, as a reference sequence, or has a specified percentage of amino acid residues, respectively, that are the same at the corresponding location within a reference sequence when the two sequences are optimally aligned. For example, an amino acid sequence that is “substantially identical” to a reference sequence has at least 50%, 60%, 70%, 75%, 80%, 85%, 90%, 95%, 96%, 97%, 98%, 99%, or 100% identity to the reference amino acid sequence. For polypeptides, the length of comparison sequences will generally be at least 5, 6, 7, 8, 9, 10, 11, 12, 13, 14, 15, 16, 17, 18, 19, 20, 25, 50, 75, 90, 100, 150, 200, 250, 300, or 350 contiguous amino acids (e.g., a full-length sequence). Sequence identity may be measured using sequence analysis software on the default setting (e.g., Sequence Analysis Software Package of the Genetics Computer Group, University of Wisconsin Biotechnology Center, 1710 University Avenue, Madison, WI 53705). Such software may match similar sequences by assigning degrees of homology to various substitutions, deletions, and other modifications.

Recombinant DNA technology now allows the design of various antibody format such as single chain antibodies (e.g., single domain), diabody, minibody, nanobody and the like which are encompassed by the present disclosure.

An “antigen-binding fragment” as referred to herein may include any suitable antigen-binding fragment known in the art. The antigen-binding fragment may be a naturally-occurring fragment or may be obtained by manipulation of a naturally-occurring antibody or by using recombinant methods. For example, an antibody fragment may include, but is not limited to a Fv, single-chain Fv (scFv; a molecule consisting of VL and VH connected with a peptide linker), Fab, F(ab′)₂, single-domain antibody (sdAb; a fragment composed of a single VL or VH), and multivalent presentations of any of these. Antibody fragments such as those just described may require linker sequences, disulfide bonds, or other type of covalent bond to link different portions of the fragments; those of skill in the art will be familiar with the requirements of the different types of fragments and various approaches and various approaches for their construction.

Antigen-binding fragments thereof of the present disclosure encompass molecules having an antigen-binding site comprising amino acid residues that confer specific binding to an antigen (e.g., one or more CDRs).

Exemplary embodiments of antigen-binding fragments disclosure thus includes without limitation (i) a Fab fragment, a monovalent fragment consisting of the V_{L}, V_{H}, C_{L} and C_{H1} domains; (ii) a F(ab′)₂ fragment, a bivalent fragment comprising two Fab fragments linked by a disulfide bridge at the hinge region; (iii) a Fd fragment consisting of the V_{H} and C_{H1} domains; (iv) a Fv fragment consisting of the V_{L} and V_{H} domains of a single arm of an antibody, (v) a dAb fragment (Ward et al., (1989) Nature 341:544-546), which consists of a V_{H} domain; and (vi) an isolated complementarity determining region (CDR), e.g., V_{H} CDR3.

Specific embodiments of antigen-binding fragments may include for example, a scFv, a Fab, a Fab′ or a (Fab′)₂.

The term “humanized antibody” encompasses fully humanized antibody (i.e., frameworks are 100% humanized) and partially humanized antibody (e.g., at least one variable region contains one or more amino acids from a human antibody, while other amino acids are amino acids of a non-human parent antibody). Typically, a “humanized antibody” contains CDRs of a non-human parent antibody (e.g., mouse, rat, rabbit, non-human primate, etc.) and frameworks that are identical to those of a natural human antibody or of a human antibody consensus. In such instance, those “humanized antibodies” are characterized as fully humanized. A “humanized antibody” may also contain one or more amino acid substitutions that have no correspondence to those of the human antibody or human antibody consensus. Such substitutions include, for example, back-mutations (e.g., re-introduction of non-human amino acids) that may preserve the antibody characteristics (e.g., affinity, specificity etc.). Such substitutions are usually in the framework region. A “humanized antibody” usually also comprise a constant region (Fc) which is typically that of a human antibody. Typically, the constant region of a “humanized antibody” is identical to that of a human antibody. A humanized antibody may be obtained by CDR grafting (Tsurushita et al, 2005; Jones et al, 1986; Tempest et al, 1991; Riechmann et al, 1988; Queen et al, 1989). Such antibody is considered as fully humanized.

The term “chimeric antibody” refers to an antibody having a constant region from an origin distinct from that of the parent antibody. The term “chimeric antibody” encompasses antibodies having a human constant region. Typically, a “chimeric antibody” is composed of variable regions originating from a mouse antibody and of a human constant region.

The term “hybrid antibody” refers to an antibody comprising one of its heavy or light chain variable region (its heavy or light chain) from a certain type of antibody (e.g., humanized) while the other of the heavy or light chain variable region (the heavy or light chain) is from another type (e.g., murine, chimeric).

Antibodies and/or antigen-binding fragments of the present disclosure may originate, for example, from a mouse, a rat or any other mammal or from other sources such as through recombinant DNA technologies. Antibodies or antigen-binding fragment of the present disclosure may include for example, a synthetic antibody, a non-naturally occurring antibody, an antibody obtained following immunization of a non-human mammal etc.

Antibodies or antigen-binding fragments thereof of the present disclosure may be isolated and/or substantially purified.

### Variant Antigen-Binding Agent

The present disclosure also encompasses variants of the antigen-binding agents, wherein said antigen-binding agents may be referred to as antigen-binding compounds, constructs, polypeptides or any compounds comprising the antibodies, antigen-binding agents or antigen-binding fragments described herein.

More particularly, the present disclosure encompasses variants of the antibodies or antigen-binding fragments, CARs and BiTEs and additionally includes ADCs, radioimmunoconjugates or any compound comprising the antigen-binding agents, antibodies or antigen-binding fragments provided herein. Variants (e.g., antibodies or antigen-binding fragments, CARs, BiTEs and the like) include those having a variation in their amino acid sequence, e.g., in one or more CDRs, in one or more framework regions and/or in the constant region. Variants (e.g., antibodies or antigen-binding fragments, CARs, BiTEs and the like) included in the present disclosure are those having, for example, similar or improved binding affinity in comparison with the original antibody or antigen-binding fragment.

Variants encompassed by the present disclosure are those which may comprise an insertion, a deletion or an amino acid substitution (conservative or non-conservative). These variants may have at least one amino acid residue in its amino acid sequence removed and a different residue inserted in its place.

More particularly, variants encompassed by the present disclosure include those having a light chain variable region and/or a heavy chain variable region having at least 80% sequence identity with the light chain variable region and/or a heavy chain variable region of the antibody or antigen-binding variant disclosed herein. The CDRs of the variant antibody may be identical to those of the antibody or antigen-binding fragments disclosed herein.

Also encompassed by the present disclosure are variants having CDRs amino acid residues that are identical and framework regions that are at least 80%, at least 90%, or at least 95% sequence identical to those of the antibody or antigen-binding fragment disclosed herein.

Conservative substitutions may be made by exchanging an amino acid residue (of a CDR, variable chain, framework region or constant region, etc.) from one of the groups listed below (group 1 to 6) for another amino acid of the same group.

Other exemplary embodiments of conservative substitutions are shown in the table below.
(group 1) hydrophobic: norleucine, methionine (Met), Alanine (Ala), Valine (Val), Leucine (Leu), Isoleucine (Ile)
(group 2) neutral hydrophilic: Cysteine (Cys), Serine (Ser), Threonine (Thr)
(group 3) acidic: Aspartic acid (Asp), Glutamic acid (Glu)
(group 4) basic: Asparagine (Asn), Glutamine (Gln), Histidine (His), Lysine (Lys), Arginine (Arg)
(group 5) residues that influence chain orientation: Glycine (Gly), Proline (Pro); and
(group 6) aromatic: Tryptophan (Trp), Tyrosine (Tyr), Phenylalanine (Phe)

Non-conservative substitutions will entail exchanging a member of one of these groups for another.

| Original | | |
|---|---|---|
| residue | Exemplary substitution | Conservative substitution |
| Ala (A) | Val, Leu, Ile | Val |
| Arg (R) | Lys, Gln, Asn | Lys |
| Asn (N) | Gln, His, Lys, Arg, Asp | Gln |
| Asp (D) | Glu, Asn | Glu |
| Cys (C) | Ser, Ala | Ser |
| Gln (Q) | Asn; Glu | Asn |
| Glu (E) | Asp, Gln | Asp |
| Gly (G) | Ala | Ala |
| His (H) | Asn, Gln, Lys, Arg, | Arg |
| Ile (I) | Leu, Val, Met, Ala, Phe, | Leu |
| | norleucine | |
| Leu (L) | Norleucine, Ile, Val, Met, | Ile |
| | Ala, Phe | |
| Lys (K) | Arg, Gln, Asn | Arg |
| Met (M) | Leu, Phe, Ile | Leu |
| Phe (F) | Leu, Val, Ile, Ala, Tyr | Tyr |
| Pro (P) | Ala | Ala |
| Ser (S) | Thr | Thr |
| Thr (T) | Ser | Ser |
| Trp (W) | Tyr, Phe | Tyr |
| Tyr (Y) | Trp, Phe, Thr, Ser | Phe |
| Val (V) | Ile, Leu, Met, Phe, Ala, | Leu |
| | norleucine | |

Percent identity is indicative of amino acids which are identical in comparison with the original peptide and which may occupy the same or similar position. Percent similarity will be indicative of amino acids which are identical and those which are replaced with conservative amino acid substitution in comparison with the original peptide at the same or similar position.

Generally, the degree of similarity and identity between variable chains has been determined herein using the Blast2 sequence program (Tatiana A. Tatusova, Thomas L. Madden (1999), “Blast 2 sequences—a new tool for comparing protein and nucleotide sequences”, FEMS Microbiol Lett. 174:247-250) using default settings, i.e., blastp program, BLOSUM62 matrix (open gap 11 and extension gap penalty 1; gapx dropoff 50, expect 10.0, word size 3) and activated filters.

A “substantially identical” sequence may comprise one or more conservative amino acid mutations, or amino acid deletions that allow for biologically functional activity to be maintained. It is known in the art that one or more conservative amino acid mutations to a reference sequence may yield a variant peptide with no substantial change in physiological, chemical, physico-chemical or functional properties compared to the reference sequence; in such a case, the reference and variant sequences would be considered “substantially identical” polypeptides.

Variants of the present disclosure therefore comprise those which may have at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity with an original sequence or a portion of an original sequence.

### Nucleic Acids, Vectors and Cells

Antibodies are usually made in cells allowing expression of the light chain and heavy chain expressed from a vector(s) comprising a nucleic acid sequence encoding the light chain and/or heavy chain.

The present disclosure therefore encompasses nucleic acids capable of encoding any of the CDRs, light chain variable regions, heavy chain variable regions, light chains, heavy chains described herein.

As used herein, the term “nucleic acid’ refers to RNA, DNA, cDNA and the like.

Due to the inherent degeneracy of the genetic code, other nucleic acid sequences that encode the same amino acid sequence may be produced and used to express the antibody or antigen-binding fragments thereof of the present disclosure. The nucleotide sequences may be engineered using methods generally known in the art in order to alter the nucleotide sequences for a variety of purposes including, but not limited to, modification of the cloning, processing, and/or expression of the gene product. DNA shuffling by random fragmentation and PCR reassembly of gene fragments and synthetic oligonucleotides may be used to engineer the nucleotide sequences. For example, oligonucleotide-mediated site-directed mutagenesis may be used to introduce mutations that create new restriction sites, alter glycosylation patterns, change codon preference, produce splice variants, and so forth.

In yet another aspect, the present disclosure relates to a vector comprising the nucleic acids described herein.

In accordance with the present disclosure, the vector may be an expression vector.

The expression vector usually contains the elements for transcriptional and translational control of the inserted coding sequence in a particular host. These elements may include regulatory sequences, such as enhancers, constitutive and inducible promoters, and 5′ and 3′ un-translated regions. Methods that are well known to those skilled in the art may be used to construct such expression vectors. These methods include in vitro recombinant DNA techniques, synthetic techniques, and in vivo genetic recombination.

The light chain variable region and the heavy chain variable region of the antibody or antigen-binding fragment thereof may be encoded by the same nucleic acid molecule (e.g., same vector) or by separate molecules (e.g., separate vectors).

The present disclosure therefore provides a set of vectors, where one of the vectors is capable of expressing the light chain or light chain variable region and the other vector is capable of expressing the heavy chain or heavy chain variable region.

Additional aspects of the disclosure relate to kits which comprising a first vial containing a nucleic acid or vector encoding the light chain or the light chain variable region of the antibody or antigen-binding fragment thereof of the present disclosure and second vial containing a nucleic acid or vector encoding the heavy chain or the heavy chain variable region of the antibody or antigen-binding fragment thereof.

In another aspect the present disclosure relates to an isolated cell which may comprise the nucleic acids, vectors, antibodies or antigen-binding fragment described herein.

The isolated cell may comprise a nucleic acid encoding a light chain variable region and a nucleic acid encoding a heavy chain variable region either on separate vectors or on the same vector. The isolated cell may also comprise a nucleic acid encoding a light chain and a nucleic acid encoding a heavy chain either on separate vectors or on the same vector.

In accordance with the present disclosure, the cell may be capable of expressing, assembling and/or secreting an antibody or antigen-binding fragment thereof.

Also, in accordance with the present disclosure, the cell may comprise and/or may express the antibody described herein.

Further in accordance with the disclosure, the cell may comprise a nucleic acid encoding a light chain variable region and a nucleic acid encoding a heavy chain variable region.

### Production of the Antibodies or Antigen-Binding Fragments in Cells

The antibodies that are disclosed herein can be made by a variety of methods familiar to those skilled in the art including hybridoma methodology or recombinant DNA methods.

Conventional hybridoma technology entails immunizing a rodent with an antigen, isolating and fusing spleen cells with myeloma cells lacking HGPRT expression and selecting hybrid cells by hypoxanthine, aminopterin and thymine (HAT) containing media. Hybridoma are screened to identify those producing antibodies that are specific for a given antigen. The hybridoma is expanded and cloned. The nucleic acid sequence of the light chain and heavy chain variable regions is obtained by standard sequencing methodology and expression vectors comprising the light chain and heavy chain nucleic acid sequence of an antibody are generated.

For recombinant expression of antibodies, host cells are transformed with a vector or a set of vectors comprising the nucleic acid sequence of the light chain and heavy chain of the antibody or antigen-binding fragment thereof (on the same vector or separate vectors).

For long-term production of recombinant proteins in mammalian systems, cell lines stably expressing proteins may be obtained. For example, nucleotide sequences able to encode any one of a light and heavy immunoglobulin chains described herein may be transformed into cell lines using expression vectors that may contain viral origins of replication and/or endogenous expression elements and a selectable or visible marker gene on the same or on a separate vector. The disclosure is not to be limited by the vector or host cell employed. In certain embodiments of the present disclosure, the nucleotide sequences able to encode any one of a light and heavy immunoglobulin chains described herein may each be ligated into a separate expression vector and each chain expressed separately. In another embodiment, both the light and heavy chains able to encode any one of a light and heavy immunoglobulin chains described herein may be ligated into a single expression vector and expressed simultaneously.

Immunological methods for detecting and measuring the expression of polypeptides are known in the art. Examples of such techniques include enzyme-linked immunosorbent assays (ELISAs), radioimmunoassays (RIAs), fluorescence activated cell sorting (FACS) or flow cytometry. Those of skill in the art may readily adapt these methodologies to the present disclosure.

Different host cells that have specific cellular machinery and characteristic mechanisms for post-translational activities (e.g., Chinese Hamster Ovary (CHO), HeLa, MDCK, HEK293, and WI-38) are available commercially and from the American Type Culture Collection (ATCC) and may be chosen to ensure the correct modification and processing of the expressed polypeptide.

Typically, antibody or antigen-binding fragments thereof are produced in CHO cells, NS0 murine myeloma cells, PER.C6® human cells.

The present disclosure relates to a method of making an antibody or an antigen-binding fragment thereof comprising expressing the light chain and heavy chain of the antibody or antigen-binding fragment of the present disclosure in cultured cells.

The method may further comprise purifying or isolating the antibody or antigen-binding fragment of the present disclosure. The method may also further comprise conjugating the antibody or antigen-binding fragment of the present disclosure to a cargo molecule such as a therapeutic or detectable moiety.

### Antibody Conjugates

The antibody or antigen-binding fragment thereof of the present disclosure may be comprised in a therapeutic or diagnostic compound, construct or composition or may be linked to a cargo molecule. Exemplary embodiments of cargo molecules include without limitation a therapeutic moiety a detectable moiety, a polypeptide (e.g., peptide, enzyme, growth factor), a polynucleotide, liposome, nanoparticle, nanowire, nanotube, quantum dot, etc.

More particularly, the antibody or antigen-binding fragment thereof of the present disclosure may be conjugated with a therapeutic moiety. The therapeutic moiety is usually attached to the antibody via a linker which may be cleavable or non-cleavable.

Included amongst the list of therapeutic moieties are cytotoxic agents, cytostatic agents, anti-cancer agents (chemotherapeutics) and radiotherapeutics (e.g. radioisotopes).

Exemplary embodiments of cytotoxic agents include, without limitation, alpha-amanitine, cryptophycin, duocarmazine, duocarmycin, chalicheamicin, deruxtecan, pyrrolobenzodiazepine (PBD), dolastatins, pseudomonas endotoxin, ricin, auristatins (e.g., monomethyl auristatin E, monomethyl auristatin F), maytansinoids (e.g., mertansine), pyrrolobenzodiazepine (PBD) and analogues.

Exemplary embodiments of radiotherapeutics include without limitation, Yttrium-90, Scandium-47, Rhenium-186, Iodine-131, Iodine-125, and many others recognized by those skilled in the art (e.g., lutetium (e.g., Lu¹⁷⁷), bismuth (e.g., Bi²¹³), copper (e.g., Cu⁶⁷), astatine-211 (211At), actinium 225 (Ac-225), etc).

Exemplary embodiments of chemotherapeutics include, without limitation, 5-fluorouracil, adriamycin, irinotecan, taxanes, carboplatin, cisplatin, etc.

The antibody or antigen-binding fragment of the present disclosure may also be conjugated with a detectable moiety (i.e., for detection or diagnostic purposes).

A “detectable moiety” comprises agents detectable by spectroscopic, photochemical, biochemical, immunochemical, chemical and/or other physical means. A detectable moiety may be coupled either directly and/or indirectly (for example via a linkage, such as, without limitation, a DOTA or NHS linkage) to antibodies and antigen-binding fragments thereof of the present disclosure using methods well known in the art. A wide variety of detectable moieties may be used, with the choice depending on the sensitivity required, ease of conjugation, stability requirements and available instrumentation. A suitable detectable moiety include, but is not limited to, a fluorescent label, a radioactive label (for example, without limitation, ¹²⁵I, In¹¹¹, Tc⁹⁹, I¹³¹ and including positron emitting isotopes for PET scanner etc.), a nuclear magnetic resonance active label, a luminescent label, a chemiluminescent label, a chromophore label, an enzyme label (for example and without limitation horseradish peroxidase, alkaline phosphatase, etc.), quantum dots and/or a nanoparticle. Detectable moiety may cause and/or produce a detectable signal thereby allowing for a signal from the detectable moiety to be detected.

### Chimeric Antigen Receptors and Other Immunotherapeutics

The sequence of the antibodies and antigen-binding fragments thereof of the present disclosure may be used to generate chimeric antigen receptors (CARs), bi-specific T-cell engagers (BiTE) or other immunotherapeutics such as for example and without limitations, bispecific killer cell engagers (BIKE), trispecific killer cell engagers (TrikE) or any immunotherapeutic compounds.

The CARs of the present disclosure may comprise for example, a) an antigen-binding domain of an antibody that specifically binds to epidermal growth factor receptor variant III (EGFRvIII), b) optionally a spacer, c) a transmembrane domain, d) optionally at least one costimulatory domain, and e) at least one intracellular signaling domain.

Chimeric antigen receptors may also comprise a hinge region or spacer which connects the antigen-binding domain and the transmembrane domain. The spacer may allow a better presentation of the antigen-binding domain at the surface of the cell.

In accordance with the present disclosure, the spacer may be optional. Alternatively, the spacer may comprise for example, between 1 to 200 amino acid residues, typically between 10 to 100 amino acid residues and more typically between 25 to 50 amino acid residues. The spacer may originate from a human protein.

In accordance with the present disclosure, the spacer or hinge region may be, for example and without limitation a CD8 hinge (e.g., mouse, human CD8) or an IgG hinge (a human immunoglobulin hinge) or combination thereof.

Exemplary embodiments of transmembrane domains include, for example and without limitation, the alpha, beta or CD3zeta chain of the T-cell receptor complex, CD28, CD27, CD3 epsilon, CD45, CD4, CD5, CD8, CD9, CD16, CD22, CD33, CD37, CD64, CD80, CD86, CD134, CD137, CD154.

In some embodiments, the transmembrane domain may include at least the transmembrane region(s) of, e.g., KIRDS2, OX40, CD2, CD27, LFA-1 (CD 11a, CD18), ICOS (CD278), 4-1BB (CD137), GITR, CD40, BAFFR, HVEM (LIGHTR), SLAMF7, NKp80 (KLRF1), NKp44, NKp30, NKp46, CD 160, CD 19, IL2R beta, IL2R gamma, IL7R a, ITGA1, VLA1, CD49a, ITGA4, IA4, CD49D, ITGA6, VLA-6, CD49f, ITGAD, CD1d, ITGAE, CD103, ITGAL, CD11a, LFA-1, ITGAM, CD11b, ITGAX, CD11c, ITGB 1, CD29, ITGB2, CD18, LFA-1, ITGB7, TNFR2, DNAM1 (CD226), SLAMF4 (CD244, 2B4), CD84, CD96 (Tactile), CEACAM1, CRT AM, Ly9 (CD229), CD160 (BY55), PSGL1, CD100 (SEMA4D), SLAMF6 (NTB-A, Lyl08), SLAM (SLAMF1, CD150, IPO-3), BLAME (SLAMF8), SELPLG (CD162), LTBR, PAG/Cbp, NKG2D, NKG2C.

A particular embodiment of transmembrane domain is the transmembrane domain of CD28.

The costimulatory domain may be, for example and without limitation, from CD28, CD27, 4-1BB, OX40, CD7, B7-1 (CD80), B7-2 (CD86), CD30, CD40, PD-1, ICOS, lymphocyte function-associated antigen-1 (LFA-1), CD2, LIGHT, NKG2C, B7-H3, a ligand that specifically binds with CD83, CDS, ICAM-1, GITR, BAFFR, HVEM (LIGHTR), SLAMF7, NKp80 (KLRF1), CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD1d, ITGAE, CD103, ITGAL, CD11a, LFA-1, ITGAM, CD11b, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Lyl08), SLAM (SLAMF1, CD150, IPO-3), BLAME (SLAMF8), SELPLG (CD162), LTBR, LAT, GADS, SLP-76, PAG/Cbp, NKp44, NKp30, NKp46, and NKG2D or a combination thereof.

The intracellular signaling domain may be, for example and without limitation, from CD3 zeta, CD3 gamma, CD3 delta, CD3 epsilon, common FcR gamma (FCERIG), FcR beta (Fc Epsilon Rib), CD79a, CD79b, Fcgamma RIIa, DAP10, or DAP12.

In order to be targeted to the secretory pathway, the chimeric antigen receptor may also comprise a signal peptide such as, for example, a signal peptide of CD28 or any other signal peptide suitable for immune cells. The signal peptide is cleaved (cleavable).

BITE, BIKE and TriKE molecules may comprise an antigen-binding domain (e.g. scFv) that specifically binds to EGFRvIII and another domain (scFv) that binds to specific immune cells including but not limited to a T-cell specific molecule (e.g., CD3) and NK-cell surface molecules (e.g. CD16). These generally comprise multiple scFvs connected in tandem by flexible linkers.

### Pharmaceutical Compositions

The present disclosure also relates to compounds, compositions, constructs and pharmaceutical compositions comprising the antibodies or antigen-binding fragments (conjugated or not) disclosed herein.

In addition to the active ingredients, a pharmaceutical composition may contain pharmaceutically acceptable carriers comprising without limitation, water, PBS, salt solutions, gelatins, oils, alcohols, and other excipients and auxiliaries that facilitate processing of the active compounds into preparations that may be used pharmaceutically. In other instances, such preparations may be sterilized.

As used herein, “pharmaceutical composition” means therapeutically effective amounts of the agent together with pharmaceutically acceptable diluents, preservatives, solubilizers, emulsifiers, adjuvant and/or carriers. A “therapeutically effective amount” as used herein refers to that amount which provides a therapeutic effect for a given condition and administration regimen. Such compositions are liquids or lyophilized or otherwise dried formulations and include diluents of various buffer content (e.g., Tris-HCl, acetate, phosphate), pH and ionic strength, additives such as albumin or gelatin to prevent absorption to surfaces, detergents (e.g., Tween 20, Tween 80, Pluronic F68, bile acid salts). Solubilizing agents (e.g., glycerol, polyethylene glycerol), antioxidants (e.g., ascorbic acid, sodium metabisulfite), preservatives (e.g., thimerosal, benzyl alcohol, parabens), bulking substances or tonicity modifiers (e.g., lactose, mannitol), covalent attachment of polymers such as polyethylene glycol to the protein, complexation with metal ions, or incorporation of the material into or onto particulate preparations of polymeric compounds such as polylactic acid, polyglycolic acid, hydrogels, etc., or onto liposomes, microemulsions, micelles, unilamellar or multilamellar vesicles, erythrocyte ghosts, or spheroplasts. Such compositions will influence the physical state, solubility, stability, rate of in vivo release, and rate of in vivo clearance. Controlled or sustained release compositions include formulation in lipophilic depots (e.g., fatty acids, waxes, oils). Also comprehended by the disclosure are particulate compositions coated with polymers (e.g., poloxamers or poloxamines). Other embodiments of the compositions of the disclosure incorporate particulate forms protective coatings, protease inhibitors or permeation enhancers for various routes of administration, including parenteral, pulmonary, nasal, oral, vaginal, rectal routes. In one embodiment the pharmaceutical composition is administered parenterally, paracancerally, transmucosally, transdermally, intramuscularly, intravenously, intradermally, subcutaneously, intraperitonealy, intraventricularly, intracranially and intratumorally.

Further, as used herein “pharmaceutically acceptable carrier” or “pharmaceutical carrier” are known in the art and include, but are not limited to, 0.01-0.1 M or 0.05 M phosphate buffer or 0.8% saline. Additionally, such pharmaceutically acceptable carriers may be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, collating agents, inert gases and the like.

For any compound, the therapeutically effective dose may be estimated initially either in cell culture assays or in animal models such as mice, rats, rabbits, dogs, or pigs. An animal model may also be used to determine the concentration range and route of administration. Such information may then be used to determine useful doses and routes for administration in humans. These techniques are well known to one skilled in the art and a therapeutically effective dose refers to that amount of active ingredient that ameliorates the symptoms or condition. Therapeutic efficacy and toxicity may be determined by standard pharmaceutical procedures in cell cultures or with experimental animals, such as by calculating and contrasting the ED₅₀ (the dose therapeutically effective in 50% of the population) and LD₅₀ (the dose lethal to 50% of the population) statistics. Any of the therapeutic compositions described above may be applied to any subject in need of such therapy, including, but not limited to, mammals such as dogs, cats, cows, horses, rabbits, monkeys, and humans.

The pharmaceutical compositions utilized in this disclosure may be administered by any number of routes including, but not limited to, oral, intravenous, intramuscular, intra-arterial, intramedullary, intrathecal, intraventricular, transdermal, subcutaneous, intraperitoneal, intranasal, enteral, topical, sublingual, or rectal means.

Additional aspects of the disclosure relate to kits which may include vial(s) containing one or more antibodies or antigen-binding fragments or antibody drug conjugates described herein.

### Methods of Use

Aspects of the disclosure comprise administering antibodies or antigen binding fragments thereof, CAR, BITE, BIKE or TriKE molecules to a subject in need.

Other aspects of the disclosure comprise administering immune cells engineered to express the CAR, BITE, BIKE or TriKE molecules to a subject in need.

The CAR, BITE, BIKE or TriKE constructs of the present disclosure may be used to re-target engineered immune cells towards EGFRvIII-positive tumors.

The engineered immune cells may be administered to a subject in need.

In accordance with an aspect of the present disclosure, immune cells are isolated from the subject, engineered to express the CAR, BITE, BIKE or TriKE construct and re-administered to the same subject.

The antibody or antigen-binding fragment thereof of the present disclosure may be used in an unconjugated form or conjugated with a therapeutic moiety in the treatment of cancer.

More particularly, the antibody or antigen-binding fragment thereof of the present disclosure may be used to inhibit the growth of tumor cells expressing EGFRvIII. Antibody drug conjugates and radioimmunoconjugates are especially contemplated for such purposes.

The present disclosure more particularly relates to a method of treating a subject having or suspected of having cancer by administering the antibody or antigen-binding fragment thereof or an antibody drug conjugate disclosed herein.

The antibody or antigen-binding fragment thereof or antibody drug conjugate may be administered as a pharmaceutical composition either alone or in combination with other anti-cancer drugs.

As used herein the term “subject” encompasses humans and animals such as non-human primates, cattle, rabbits, mice, rats, sheep, goats, horses, birds, etc. The term “subject” particularly encompasses humans.

Subjects in need which would benefit from treatment include humans having tumor cells expressing EGFRvIII. More particularly, the antibody or antigen-binding fragments thereof or antibody drug conjugate may be administered to a subject suspected of having glioblastoma multiforme (GBM). Subjects in need also encompass those having or suspected of having carcinomas, such as those from breast, head and neck or oral origin.

The term “treatment” for purposes of this disclosure refers to both therapeutic treatment and prophylactic or preventative measures, wherein the object is slow down (lessen) the targeted pathologic condition or disorder. Those in need of treatment include those already with the disorder as well as those prone to have the disorder or those in whom the disorder is to be prevented. Particularly, subjects in need include subjects with an elevated level of one or more cancer markers.

Alternatively, in order to carry out the methods of the present disclosure and as known in the art, the antibody or antigen-binding fragment of the present disclosure (conjugated or not) may be used in combination with a second molecule (e.g., a secondary antibody, etc.) which is able to specifically bind to the antibody or antigen-binding fragment of the present disclosure and which may carry a desirable detectable, diagnostic or therapeutic moiety.

The antibody or antigen-binding fragment thereof of the present disclosure may be used in an unconjugated form or conjugated with a detectable moiety in assays or methods involving detection of EGFRvIII.

Methods of treating subject having a cancer associated with EGFRvIII expression are particularly contemplated. Such method may comprise administering an antigen-binding agent disclosed herein or cells expressing such antigen-binding agent.

In an exemplary embodiment, the method may comprise administering an antibody-drug conjugate.

In another exemplary embodiment, the method may comprise administering cells expressing a chimeric antigen receptor, a bi-specific T-cell engager, a bispecific killer cell engager or a trispecific killer cell engager.

Another aspect of the disclosure relates a method for detecting EGFRvIII, the method may comprise contacting a cell expressing EGFRvIII, or a sample (biopsy, a body fluid such as serum, plasma, urine etc.) comprising or suspected of comprising EGFRvIII with the antibody or antigen-binding fragments described herein and measuring binding. The sample may originate from a mammal (e.g., a human) which may have cancer (e.g., glioblastoma multiforme or carcinoma) or may be suspected of having such cancer. The sample may be a tissue sample obtained from the mammal or a cell culture supernatant.

In accordance with the disclosure the sample may be a serum sample, a plasma sample, a blood sample or ascitic fluid obtained from the mammal.

Further scope, applicability and advantages of the present disclosure will become apparent from the non-restrictive detailed description given hereinafter. It should be understood, however, that this detailed description, while indicating exemplary embodiments of the disclosure, is given by way of example only, with reference to the accompanying drawings.

### EXAMPLES

### Example 1: Design of Humanized V-Region Sequences

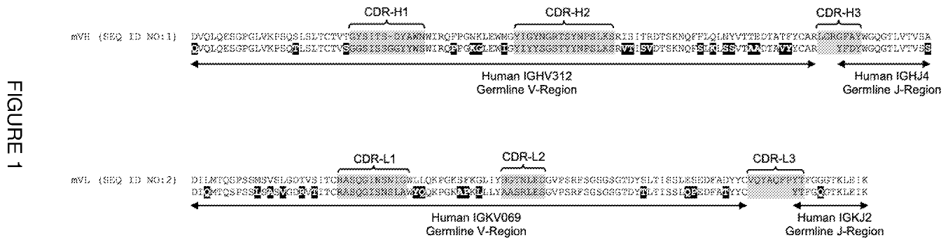

The variable region of the murine anti-EGFR vIII-specific antibody 4E11 was humanized here. The corresponding murine VH and VL amino-acid sequences are listed in the Sequence Listing Table under SEQ ID NO: 1 and SEQ ID NO:2, respectively, and named mVH and mVL, respectively. The CDR loops embedded in these V-region sequences are delineated in FIG. 1 according to the Kabat definition, except for the CDR-H1 loop which is delineated according to the combined Kabat and Chothia definitions (Wu and Kabat, 1970; Kabat and Wu, 1991; Chothia and Lesk, 1987; Al-Lazikani et al., 1997). This definition formed the basis for the humanization of framework regions (FR) of mVH and mVL by the CDR grafting approach (Riechmann et al., 1988).

The CDR grafting was done by stitching the three CDR segments of the source mVH sequence to the four FR segments of a human VH template sequence, in the appropriate order; and the three CDR segments of the source mVL sequence to the four FR segments of a human VL template, in the appropriate order. We first identified that the closest human germline VH and VL families for the mVH and mVL sequences of the 4E11 antibody are the human V_{H}-4 family and the human Vk-1 family, respectively. The VBASE2 human germline database (Retter et al, 2005) was then used identify several human V-gene and human J-gene sequences that scored highly in terms of sequence homology to the FRs of the parental murine sequences. The final human germline template sequence selected for humanization are shown in FIG. 1 aligned to the corresponding murine sequences, and they are: human IGHV312 (V-segment) and human IGHJ4 (J-segment) for the mVH sequence, and human IGKV069 (V-segment) and human IGKJ2 (J-segment) for the mVL sequence. Given these templates, there are 20 mouse-to-human amino-acid mutations required to fully humanize the mVH FRs and 16 mouse-to-human amino-acid mutations required to fully humanize the mVL FRs (see highlighted amino-acids in FIG. 1). Mutating all these 36 amino-acids will lead to a 100% humanized V-region, which is represented by the hVH1 (also comprised in hH1) variant with SEQ ID NO: 5 and the hVL1 (also comprised in hL1) variant with SEQ ID NO: 8.

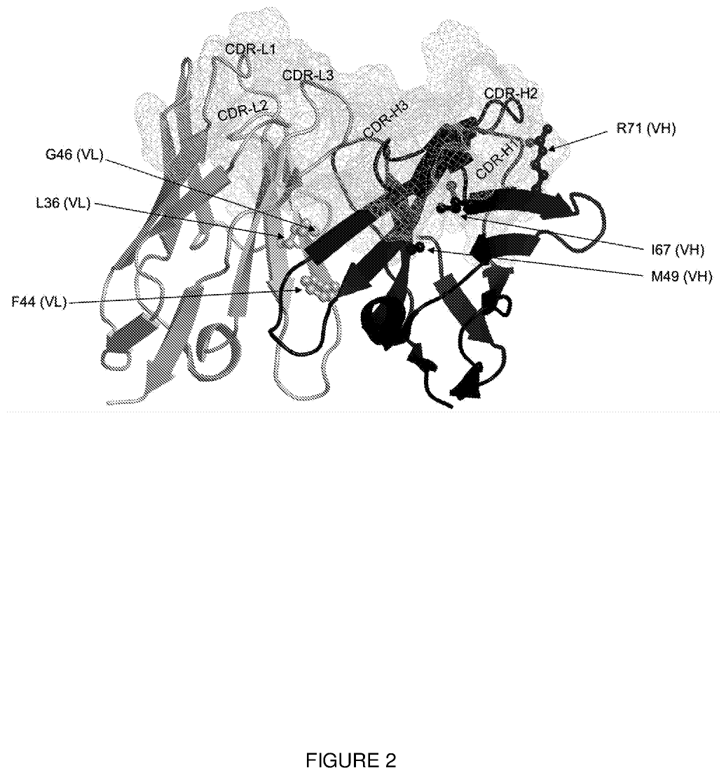

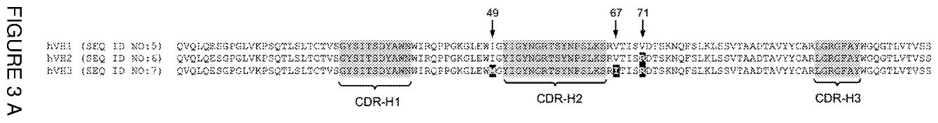

In many cases, it has been observed that the 100% humanized variants possess somewhat reduced antigen binding relative to the parental murine variant. To mitigate the risk of antigen binding affinity loss, “back-mutations” to the original parental murine amino-acid residues were designed. There are amino-acid residues in the source antibody framework that may directly or indirectly impact antigen binding. To assist the identification of back-mutations, a 3D model of murine 4E11 variable region was built by homology modeling using a suitable crystal structure as template (Protein Data Bank code: 3G5V). A rendering of this molecular model is shown in FIG. 2. Based on close visual examination and detailed structural analysis of this structural model, the following back-mutations were selected: M49, I67 and R71 in the heavy chain; and L36, F44 and G46 in the light chain (Kabat numbering is used throughout); these back-mutation amino-acids are highlighted in FIG. 2.

In the VH, the back-mutation at position 71, where the Arginine amino-acid side-chain directly supports the conformation of CDR-H1 and also CDR-H2, is likely implicated in antigen binding. The non-conservative R71V mutation required for humanization at this position may introduce significant structural changes in the CDR. Hence, implementation of this mutation (R71) led to the humanized variant hVH2 with SEQ ID NO: 6. Hydrophobic residues at the more buried positions 49 and 67 are of a lesser concern, but still significant in order to maintain the correct packing that directly supports the conformation of the CDR-H2 loop. Although the humanization at these two positions would require conservative mutations M491 and 167V, the correct packing would be impaired. Therefore, implementation of these back-mutations (M49, 167) in addition to the R71 back-mutation described earlier led to the humanized variant hVH3 with SEQ ID NO: 7.

In the VL, the back-mutations appear in the form of a tandem of two amino-acids that are in close spatial proximity, L36 and G46, which directly support the conformation of the hypervariable CDR-H3 loop from the pairing chain as well as CDR-L2 from the same chain; which are very likely implicated directly in antigen binding. A striking structural feature is that the humanization required at these two positions, L36Y and G46L, cannot be accommodated sterically due to a significant increase in size at a relatively buried location, which will likely lead to significant structural alterations. Hence, implementation of these back-mutations (L36,G46) led to the humanized variant hVL2 with SEQ ID NO: 9. A further back-mutation is identified at the murine amino-acid F44, which is also clustered around the aforementioned tandem back-mutation amino-acids, and at which position humanization requires mutation to the more conformationally-restricted and smaller Proline amino-acid. Therefore, implementation of this back-mutation (F44) in addition to the tandem back-mutation described earlier (L36, G46) led to the humanized variant hVL3 with SEQ ID NO: 10.

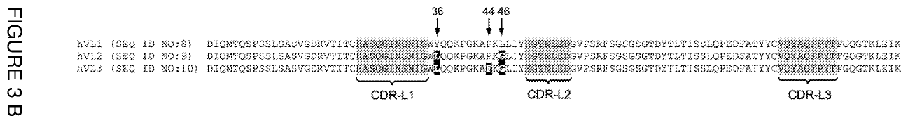

The designed humanized V-region sequences for the 4E11 antibody are aligned in FIG. 3. Further in silico analysis indicated the absence of predicted T-cell epitopes for all these humanized sequences (Dhanda et al., 2018).

### Example 2: Recombinant Production and Purification of Full-Length Antibodies

The chimeric and humanized VH and VL regions of the described in Example 1 were cloned as fusions with human IgG4 constant regions (human IgG4 heavy chain and human kappa light chain, respectively) into the pTT5™ vector, thereby generating chimeric antibodies. The human IgG4 heavy chain sequence was mutated at position 228 from a Serine amino-acid residue to a Proline residue (i.e., S228P mutation) in order to increase the stability of the IgG4 homodimer. The C-terminal Lysine amino-acid residue of the heavy chain was deleted in order to reduce the heterogeneity due to the partial clipping of this C-terminal Lysine as post-translation modification. The resulting full-length heavy-chain and light-chain chimeric sequences, cH and cL, respectively, are given in the Sequence Listing Table with SEQ ID NO: 11 and SEQ ID NO: 12, respectively. The resulting full-length heavy-chain humanized variant sequences, hH1, hH2 and hH3, are given in the Sequence Listing Table with SEQ ID NO: 13, SEQ ID NO: 14 and SEQ ID NO: 15, respectively. The resulting full-length light-chain humanized variant sequences hL1, hL2 and hL3, are given in the Sequence Listing Table with SEQ ID NO: 16, SEQ ID NO: 17 and SEQ ID NO: 18, respectively. All light-chain sequences comprise an optional signal peptide MVLQTQVFISLLLWISGAYG (SEQ ID NO: 20) at the N-terminus, while heavy-chain sequences comprise the signal peptide MDWTWRILFLVAAATGTHA (SEQ ID NO: 19) at the N-terminus. These are non-limiting exemplary sequences of signal peptides and one skilled in the art can select other suitable signal peptides for recombinant production of these antibody variants.

The chimeric antibody cH-cL and the 9 humanized antibodies covering all possible combinations between the 3 humanized heavy chains and 3 humanized light chains (hH1-hL1, hH1-hL2, hH1-hL3, hH2-hL1, hH2-hL2, hH2-hL3, hH3-hL1, hH3-hL2, hH3-hL3) were produced recombinantly in CHO cells. In this example, production was carried out at 25-mL scale using the CHO55E1 cells at 32° C. Briefly, CHO cells were transiently transfected with heavy-chain and light-chain constructs (1:1 ratio). Conditioned medium was harvested on day 8 post-transfection, when viable cell density was 1-3×10⁷ cells/mL and cell viability was 84-93% as determined by direct counting of cell samples with a Cedex automated cell counting system (Roche Innovatis). All antibody variants were well expressed by the transiently transfected CHO cells.

Purifications from cell-culture supernatants were performed by protein-A affinity chromatography using 1-mL HiTrap MabSelect SuRe columns (GE Healthcare) in parallel on a Protein Maker system. Column equilibration and washing were done in DPBS (GE Healthcare Life Sciences). Linear flow rate for binding was set at ˜45 cm/h (0.3 mL/min) to get a residence time of ˜3.3 minutes. Elution was done with citrate buffer 0.1 M, pH 3.0, and neutralization was done in 10% (v/v) 1 M HEPES. Fractions containing the antibodies were pooled and the citrate buffer was exchanged against DPBS was done ZebaSpin TK MWCO columns (ThermoFisher Scientific). Purified antibodies were sterilized by filtration through 0.2 μm filters. Ultra-high performance liquid chromatography-size exclusion chromatography (UPLC-SEC) was used to assess the purity of all eluates. Purity was above 95% for 5 humanized variants and between 89-95% for the chimeric variant and the remaining 3 humanized variants. Selected peak fractions were concentrated by ultrafiltration using Vivaspin® Turbo centrifugal concentrator with a membrane molecular weight cut off of 30 kDa (GE Healthcare Life Sciences) at room temperature following the manufacturer's instructions. During the process, the protein concentration was monitored on a NanoDrop™ 2000 spectrophotometer (ThermoFisher Scientific) using absorbance at 280 nm and the calculated specific extinction coefficient of each variant. Variants with purities below 95% were further purified by preparative SEC on Superdex 200 10/30 columns (GE Healthcare Life Sciences). Final products were analyzed by SDS-PAGE and UPLC-SEC and had final purities of 99-100%.

### Example 3: Thermal Stability Measurements by Differential Scanning Calorimetry

Differential scanning calorimetry (DSC) was used to determine the thermal transition midpoints (Tₘ) of 4E11 humanized and chimeric antibody variants using a Malvern MicroCal DSC system (Malvern Instruments). Samples in DPBS buffer were diluted in the DPBS buffer to a final concentration of 0.4 mg/mL. Thermal denaturation was carried out by increasing the temperature from 20° C. to 100° C. at a rate of 60° C./h, with feedback mode/gain set at “low”, filtering period of 8 s, pre-scan time of 3 min, and under 70 psi of nitrogen pressure. All data were analyzed with Origin 7.0 software (OriginLab) with manual baseline assignment and Tₘ determined using a fit to three transitions.

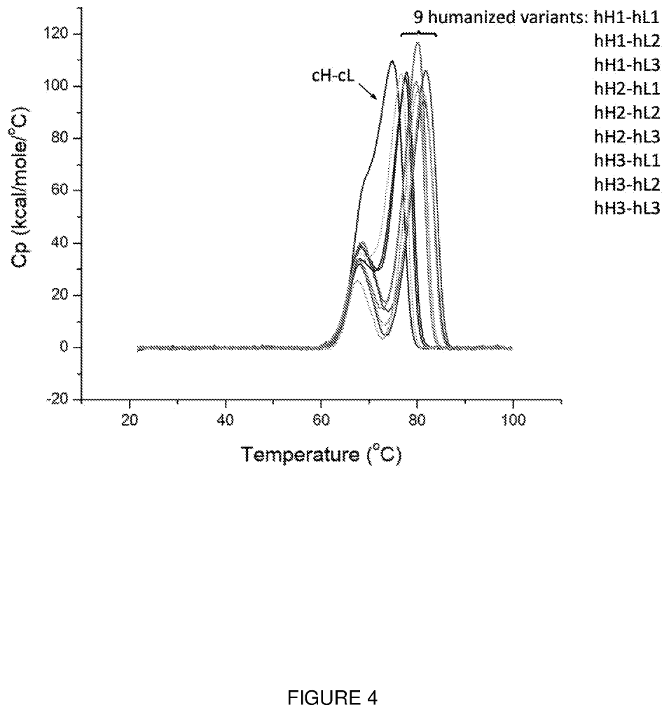

Results are shown in FIG. 4 in terms of thermograms and Table 1 in terms melting temperatures obtained from the integrated thermograms. All humanized variants advantageously showed improved thermal stabilities relative to the chimeric variant (FIG. 4). It is apparent from the thermogram of the chimeric antibody has a completely different shape consisting of a single peak with multiple shoulders, whereas the humanized variants have two well-separated peaks with the second peak having a shoulder and being shifted towards higher temperature.

Since the sequence difference between these variants occur in the V-region that is part of the Fab fragment, of particular interest is the observed advantageous increased stabilities of the Fab domains for all humanized variants relative to the chimeric variant, as reflected by higher Tₘ2 values (Table 1). These improvements in the stabilities of the Fab fragment upon humanization range between 1.1° C. to 6.2° C. One of skill in this art would understand that antibody humanization typically results in a loss of thermal stability and/or does not lead to improvements in stability. It is therefore advantageous and unexpected that the now provided humanized variants exhibit improvements in thermal stability.

The stability of the CH3 domain, as reflected by the Tₘ2 values (Table 1) was also improved for all humanized variants relative to the chimeric variants, with Tₘ3 increases ranging between 1.0° C. and 6.6° C. This finding is very interesting and surprising, since the CH3 domain sequence is identical between all these variants. This unexpected finding suggest an indirect allosteric effect of the humanization mutations designed in this invention on the overall stability of the 4E11 antibody, even at sites that are remote from the introduced humanization mutations.

Further structure-property relationship analysis shows that the humanized variants having the hL1 light-chain variant exhibit minimal improvements in stability relative to the chimeric variant, whereas those having the hL2 light-chain variant exhibit the largest improvements in stability relative to the chimeric variant. This applies to both Tₘ2 (Fab melting) and Tₘ3 (CH3 melting). No significant dependence of folding stability can be detected with respect to the sequence humanized heavy-chain variant.

TABLE 1
Thermal stability determined by differential scanning calorimetry.

| | | Tₘ1 | Tₘ2 | Tₘ3 |
|---|---|---|---|---|
| 4E11 variant | SEQ ID NOs | (° C.) | (° C.) | (° C.) |
| cH-cL | 11; 12 | 69.3 | 73.7 | 76.0 |
| hH1-hL1 | 13; 16 | 68.7 | 76.0 | 78.2 |
| hH1-hL2 | 13; 17 | 68.2 | 79.6 | 82.1 |
| hH1-hL3 | 13; 18 | 68.9 | 78.2 | 80.6 |
| hH2-hL1 | 14; 16 | 68.7 | 75.6 | 77.8 |
| hH2-hL2 | 14; 17 | 68.8 | 79.9 | 82.6 |
| hH2-hL3 | 14; 18 | 67.9 | 78.5 | 80.8 |
| hH3-hL1 | 15; 16 | 68.7 | 74.8 | 77.0 |
| hH3-hL2 | 15; 17 | 68.0 | 79.5 | 82.0 |
| hH3-hL3 | 15; 18 | 68.3 | 78.0 | 80.3 |

### Example 4: Evaluation of Apparent Affinity by Flow Cytometry

The binding properties of the recombinant purified chimeric and 9 humanized variants of the 4E11 anti-EGFRvIII monoclonal antibodies were assessed for their binding affinity and specificity by flow cytometry in a dose-dependent binding curve on human glioblastoma cell lines U87MG overexpressing wild-type EGFR (U87MG-EGFR wt or U87 WT) and U87MG overexpressing EGFRvIII mutation (42-7 deletion mutation of EGFR; U87MG-EGFRvIII or U87vIII). Cells overexpressing full length wt EGFR or EGFRvIII were obtained from the laboratory of W. Cavanee (Ludwig Institute for Cancer Research, University of California at San Diego). Cells were grown in DMEM high glucose medium containing 10% FBS and 400 μg/ml G418.

Prior to analysis, cells were plated such that they were not more than 80% confluent on the day of analysis. Unless otherwise stated, all media are kept are 4° C. and all incubations are performed on wet ice. Cells were washed in PBS, harvested by the addition of cell dissociation buffer (Sigma), centrifuged and resuspended in complete medium at a cell density of 2×10⁶ cells/mL. Fifty μL/well of cells were distributed in a polypropylene v-bottom 96 well plate and serial 1/3 dilutions of purified mAbs starting at 100 nM were added and incubated for 2 hours. Cells were washed twice by centrifugation and further incubated with a FITC labeled F(ab′)₂ goat anti-mouse antibody (Fc specific, #115-096-071, Jackson Immunoresearch, Cedarlane) for an hour. Cells were washed and re-suspended in medium containing Propidium iodide to exclude dead cells from analysis. Samples were filtered through a 60 μm nylon mesh filter plate (Millipore) to remove cell aggregates. Flow cytometry analyses were performed on 2,000 viable single-cells events gated on forward scattering, side scattering parameters and propidium iodide dye exclusion using a BD-LSRFortessa flow cytometer (Becton-Dickinson Biosciences) and a standard filter set using BD FACSDiva™ acquisition software, according to manufacturer's instructions.

Specific detection of antibody binding was calculated as the mean fluorescent intensity of binding to each primary antibody after background level subtraction of the mean fluorescent intensity of binding of human IgG ChromPure (Jackson Immuno #009-000-003 used as negative control). The data were analyzed with GraphPad Prism v 8.4.3 software using one-site specific binding with Hill slope non-linear regression curve fit model to determine Bₘₐₓ (maximum specific binding) and K_{D-app} (concentration needed to achieve a half-maximum binding at equilibrium) for each antibody variant tested. The model used was according to formula:

Y=Bₘₐₓ*X^{h}/(K_{D}^{h}+X^{h}), where:
Bₘₐₓ is the maximum specific binding, in the same unit as Y;
K_{D} is the ligand concentration needed to achieve half maximum binding at equilibrium, expressed in the same unit as X; and
h is a variable “h” representing the Hill slope.

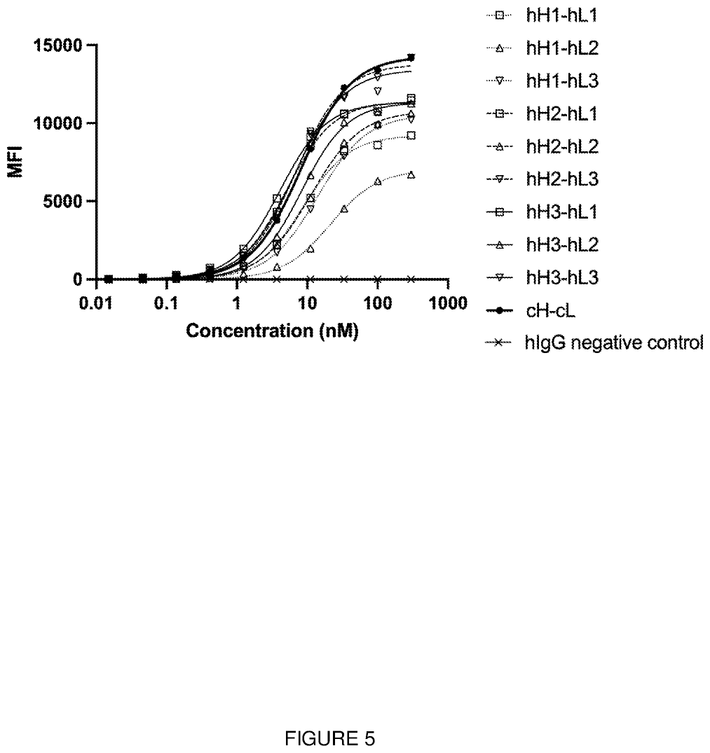

FIG. 5 shows exemplary results of flow cytometry experiments determining the binding properties of anti-EGFRvIII monoclonal antibodies to cell surface-expressed EGFRvIII. Corresponding K_{D-app} values averaged over multiple experiments are listed in Table 2. All humanized anti-EGFRvIII antibodies showed strong and comparable binding to cells overexpressing the EGFRvIII variant. Even though antibody humanization typically results in a loss of antigen binding affinity, in this case, out of the 9 humanized variants, 3 humanized variants have statistically significantly improved binding (hH2-hL1, hH2-hL3 and hH3-hL1), 4 humanized variants have the same binding within statistical errors (hH1-hL1, hH2-hL2, hH3-hL2 and hH3-hL3) and 2 humanized variants have only slightly decreased binding within a 2-3-fold range (hH1-hL2 and hH1-hL3), relative to the chimeric variant (cH-cL). Hence, these data represent unusual and unexpected humanization successes in terms of the retention of, and even improvements from, the parental binding affinity for most humanized variants designed. The humanized variants now provided exhibit unexpected and advantageous increased thermal stability and in most cases unexpected and advantageous improved binding affinity relative to the parental antibody. This is impressive taking into account the large number of mutations introduced in each of the two Fab arms of these full-length humanized variants (ranging between 30 to 36 mutations per Fab arm relative to the chimeric variant). A cursory structure-activity relationship indicates that for each humanized heavy chain, the hL1 light-chain variant has the strongest binding and the hL2 light-chain variant has the weakest binding; and that for each humanized light chain, the hH3 heavy-chain variant has the strongest binding and the hH1 heavy-chain has the weakest binding.

TABLE 2
Apparent binding affinity (K_{D-app}) of anti-EGFR vIII
humanized and chimeric 4E11 antibodies to the U87-MG-EGFR-vIII
cell line determined by flow cytometry.

K_{D-app} (nM)

| 4E11 variant | SEQ ID NOs | Mean | SD |
|---|---|---|---|
| cH-cL | 11; 12 | 8.8 | 1.3 |
| hH1-hL1 | 13; 16 | 9.7 | 1.5 |
| hH1-hL2 | 13; 17 | 23.0 | 2.6 |
| hH1-hL3 | 13; 18 | 15.7 | 3.0 |
| hH2-hL1 | 14; 16 | 5.8 | 1.1 |
| hH2-hL2 | 14; 17 | 10.5 | 1.4 |
| hH2-hL3 | 14; 18 | 7.2 | 0.3 |
| hH3-hL1 | 15; 16 | 4.9 | 0.7 |
| hH3-hL2 | 15; 17 | 7.9 | 0.3 |
| hH3-hL3 | 15; 18 | 7.1 | 0.6 |

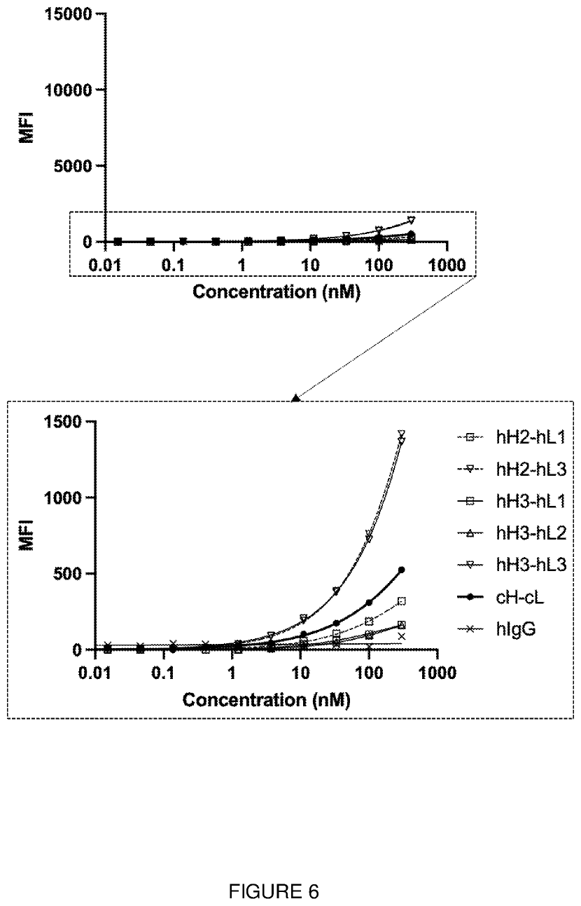

In order to confirm the specificity of the humanized variants against the cells expressing the wild-type EGFR, five of the humanized variants with the best binding to the cells expressing the EGFR vIII (hH2-hL1, hH2-hL3, hH3-hL1, hH3-hL2 and hH3-hL3) were tested by flow cytometry U87MG EGFR wt cells. The results shown in FIG. 6 and Table 3 indicate that the excellent binding specificity of the chimeric variant has been retained in the tested humanized variants, and even further improved in the case hH2-hL1, hH3-hL1 and hH3-hL3 humanized antibodies. This advantageous specificity may lead to even lower toxicities of these humanized 4E11 antibody variants of the present invention due to negligible binding to normal cells and tissues where wild-type RGFR is expressed.

TABLE 3
Binding specificity of anti-EGFR vIII humanized and chimeric
4E11 antibodies against wild-type EGFR determined by flow
cytometry using the U87-MG-EGFR-wt cell line.

| | | | Bₘₐₓ @ 300 nM |
|---|---|---|---|
| 4E11 variant | SEQ ID NOS | K_{D-app} (nM) | (MFI) |
| cH-cL | 11; 12 | >300 | 525 |
| hH2-hL1 | 13; 16 | >300 | 319 |
| hH2-hL3 | 13; 17 | >300 | 1418 |
| hH3-hL1 | 13; 18 | >300 | 164 |
| hH3-hL2 | 14; 16 | >300 | 160 |
| hH3-hL3 | 14; 17 | >300 | 1367 |

As now shown, the humanized antibodies of the present invention exhibit excellent characteristics in terms of thermal stability and antigen binding and specificity, which are unexpected and surprising given the observed improvements of these properties relative to the chimeric antibody having the murine V-region of the parental 4E11 anti-EGFRvIII antibody.

The embodiments and examples described herein are illustrative and are not meant to limit the scope of the disclosure as claimed. Variations of the foregoing embodiments, including alternatives, modifications and equivalents, are intended by the inventors to be encompassed by the claims. Citations listed in the present application are incorporated herein by reference.

### REFERENCES

All patents, patent applications and publications referred to throughout the application are incorporated herein by reference.
Abhinandan, K R and Martin, ACR. Analysis and improvements to Kabat and structurally correct numbering of antibody variable domains. 2008; Mol Immunol, 45, 3832-3839.
Cleary, J M et al., Invest New Drugs, 33 (3), pp. 671-8, 2015;
Chothia C, Lesk A M. Canonical structures for the hypervariable regions of immunoglobulins. J Mol Biol. 1987 Aug. 20; 196 (4): 901-17.
Elvin A. Kabat, Tai Te Wu, Carl Foeller, Harold M. Perry, Kay S. Gottesman, Sequences of Proteins of Immunological Interest, Vol. 1 DIANE Publishing, 1992-2719.
Feldhaus M J et al., 2003 Nat Biotechnol. 2003 February; 21 (2): 163-70.
Gan H K, Cvrljevic A N, Johns T G. The epidermal growth factor receptor variant III (EGFRvIII): where wild things are altered. FEBS J. 2013 280): 5350-70.
Hamblett K. J, et al., Molecular Cancer Therapeutics, Vol. 14 (7), pp. 1614-24, 2015.
Johnson G, Wu T T. The Kabat database and a bioinformatics example. Methods Mol Biol. 2004; 248:11-25.
Jones P T, Dear P H, Foote J, Neuberger M S, Winter G (1986) Nature 321, 522-525.
Kabat E A, Wu T T. Identical V region amino acid sequences and segments of sequences in antibodies of different specificities. Relative contributions of V_{H} and V_{L} genes, minigenes, and complementarity-determining regions to binding of antibody-combining sites. J Immunol. 1991; 147:1709-19.
Mendelsohn J, et al., Clin Cancer Res. 2015 Jan. 15; 21 (2): 227-9.
Reilly, EB., Molecular Cancer Therapeutics, Vol. 14 (5), pp. 1141-51, 2015.201
Jones P T, Dear P H, Foote J, Neuberger M S, Winter G (1986) Nature 321, 522-525
Riechmann L, Clark M, Waldmann H, Winter G (1988) Nature 332, 323-327.
Sato J D, et al., Mol. Biol. Med. 1:511-529, 1983.
Tempest P R, Bremmer P, Lambert M, Taylor G, Furze J M, Carr F J, Harris W J (1991) Biotechnology 9, 266-271.
Tsurushita N, Hinton, RP, Kumar S (2005) Design of humanized antibodies: From anti-Tac to Zenapax. Methods 36, 69-83.
Queen C, Schneider W P, Selick H E, Payne P W, Landolfi N F, Duncan J F, Avdalovic N M, Levitt M, Junghans R P, Waldmann T A (1989) Proc Natl Acad Sci USA 86, 10029-10033.
Tempest P R, Bremmer P, Lambert M, Taylor G, Furze J M, Carr F J, Harris W J (1991) Biotechnology 9, 266-271.
U.S. Pat. No. 7,736,644.
U.S. Pat. No. 4,943,533 dated Jul. 24 1990.
Yano S, Kondo K, Yamaguchi M, Richmond G, Hutchison M, Wakeling A, Averbuch S, Wadsworth P. Distribution and function of EGFR in human tissue and the effect of EGFR tyrosine kinase inhibition. Anticancer Res. 2003 September-October; 23 (5A): 3639-50.
Cleary J M, Reardon D A, Azad N, Gandhi L, Shapiro G I, Chaves J, Pedersen M, Ansell P, Ames W, Xiong H, Munasinghe W, Dudley M, Reilly E B, Holen K, Humerickhouse R. A phase 1 study of ABT-806 in subjects with advanced solid tumors. Invest New Drugs. 2015 June; 33 (3): 671-8 2015
Panousis C, Rayzman V M, Johns T G, Renner C, Liu Z, Cartwright G, Lee F T, Wang D, Gan H, Cao D, Kypridis A, Smyth F E, Brechbiel M W, Burgess A W, Old L J, Scott A M. Engineering and characterisation of chimeric monoclonal antibody 806 (ch806) for targeted immunotherapy of tumours expressing de2-7 EGFR or amplified EGFR. Br J Cancer. 2005 Mar. 28; 92 (6): 1069-77.
Wu T T, Kabat E A. An analysis of the sequences of the variable regions of Bence Jones proteins and myeloma light chains and their implications for antibody complementarity. J Exp Med 1970; 132 (2): 211-250.
Al-Lazikani B, Lesk A M, Chothia C. Standard conformations for the canonical structures of immunoglobulins. J Mol Biol 1997; 273 (4): 927-948.
Retter I, Althaus H H, Munch R, Muller W. VBASE2, an integrative V gene database. Nucl Acids Res 2005; 33 (suppl_1):D671-D674
Dhanda S K, Grifoni A, Pham J, Vaughan K, Sidney J, Peters B, Sette A. Development of a strategy and computational application to select candidate protein analogues with reduced HLA binding and immunogenicity. Immunology 2018; 153 (1): 118-132.

SEQUENCE TABLE

| SEQ | | | |
|---|---|---|---|
| ID NO | Name | Description | Sequence |
| 1 | mVH | Murine 4E11 | DVQLQESGPGLVKPSQSLSLTCTVTGYSITSDYAWNWIRQFPGNKLEWMGYIGYNG |
| | | VH region | RTSYNPSLKSRISITRDTSKNQFFLQLNYVTTEDTATFYCARLGRGFAYWGQGTLV |
| | | | TVSA |
| 2 | mVL | Murine 4E11 | DILMTQSPSSMSVSLGDTVSITCHASQGINSNIGWLLQKPGKSFKGLIYHGTNLED |
| | | VL region | GVPSRFSGSGSGTDYSLTISSLESEDFADYYCVQYAQFPYTFGGGTKLEIK |
| 3 | hVHx | Consensus | QVQLQESGPGLVKPSQTLSLTCTVSGYSITSDYAWNWIRQPPGKGLEWX1GYIGYN |
| | | humanized | GRTSYNPSLKSRX2TISX3DTSKNQFSLKLSSVTAADTAVYYCARLGRGFAYWGQG |
| | | 4E11 VH | TLVTVSS |
| | | region | where X₁ = I or M, X₂ = V or I, and X₃ = V or R |
| 4 | hVLx | Consensus | DIQMTQSPSSLSASVGDRVTITCHASQGINSNIGWX4QQKPGKAX5KX6LIYHGTNL |
| | | humanized | EDGVPSRFSGSGSGTDYTLTISSLQPEDFATYYCVQYAQFPYTFGQGTKLEIK |
| | | 4E11 VL | where X₄ = Y or L, X₅ = P or F, and X₆ = L or G |
| | | region | |
| 5 | hVH1 | Humanized | QVQLQESGPGLVKPSQTLSLTCTVSGYSITSDYAWNWIRQPPGKGLEWIGYIGYNG |
| | | 4E11 VH | RTSYNPSLKSRVTISVDTSKNQFSLKLSSVTAADTAVYYCARLGRGFAYWGQGTLV |
| | | region | TVSS |
| | | variant 1 | |
| 6 | hVH2 | Humanized | QVOLQESGPGLVKPSQTLSLTCTVSGYSITSDYAWNWIRQPPGKGLEWIGYIGYNG |
| | | 4E11 VH | RTSYNPSLKSRVTISRDTSKNQFSLKLSSVTAADTAVYYCARLGRGFAYWGQGTLV |
| | | region | TVSS |
| | | variant 2 | |
| 7 | hVH3 | Humanized | QVQLQESGPGLVKPSQTLSLTCTVSGYSITSDYAWNWIRQPPGKGLEWMGYIGYNG |
| | | 4E11 VH | RTSYNPSLKSRITISRDTSKNQFSLKLSSVTAADTAVYYCARLGRGFAYWGQGTLV |
| | | region | TVSS |
| | | variant 3 | |
| 8 | hVL1 | Humanized | DIQMTQSPSSLSASVGDRVTITCHASQGINSNIGWYQQKPGKAPKLLIYHGTNLED |
| | | 4E11 VL | GVPSRFSGSGSGTDYTLTISSLOPEDFATYYCVQYAQFPYTFGQGTKLEIK |
| | | region | |
| | | variant 1 | |
| 9 | hVL2 | Humanized | DIQMTQSPSSLSASVGDRVTITCHASQGINSNIGWLQQKPGKAPKGLIYHGTNLED |
| | | 4E11 VL | GVPSRFSGSGSGTDYTLTISSLOPEDFATYYCVQYAQFPYTFGQGTKLEIK |
| | | region | |
| | | variant 2 | |
| 10 | hVL3 | Humanized | DIQMTQSPSSLSASVGDRVTITCHASQGINSNIGWLQQKPGKAFKGLIYHGTNLED |
| | | 4E11 VL | GVPSRFSGSGSGTDYTLTISSLOPEDFATYYCVQYAQFPYTFGQGTKLEIK |
| | | region | |
| | | variant 3 | |
| 11 | cH | Chimeric | DVQLQESGPGLVKPSQSLSLTCTVTGYSITSDYAWNWIRQFPGNKLEWMGYIGYNG |
| | | 4E11 heavy | RTSYNPSLKSRISITRDTSKNOFFLOLNYVTTEDTATFYCARLGRGFAYWGQGTLV |
| | | chain on | TVSAASTKGPSVFPLAPCSRSTSESTAALGCLVKDYFPEPVTVSWNSGALTSGVHT |
| | | human | FPAVLQSSGLYSLSSVVTVPSSSLGTKTYTCNVDHKPSNTKVDKRVESKYGPPCPP |
| | | IgG4(S228P) | CPAPEFLGGPSVFLFPPKPKDTLMISRTPEVTCVVVDVSQEDPEVQFNWYVDGVEV |
| | | background | HNAKTKPREEQFNSTYRVVSVLTVLHQDWLNGKEYKCKVSNKGLPSSIEKTISKAK |
| | | | GQPREPQVYTLPPSQEEMTKNQVSLTCLVKGFYPSDIAVEWESNGOPENNYKTTPP |
| | | | VLDSDGSFFLYSRLTVDKSRWQEGNVFSCSVMHEALHNHYTQKSLSLSLG |
| 12 | cL | Chimeric | DILMTQSPSSMSVSLGDTVSITCHASQGINSNIGWLLQKPGKSFKGLIYHGTNLED |
| | | 4E11 light | GVPSRFSGSGSGTDYSLTISSLESEDFADYYCVQYAQFPYTFGGGTKLEIKRTVAA |
| | | chain on | PSVFIFPPSDEQLKSGTASVVCLLNNFYPREAKVOWKVDNALQSGNSQESVTEQDS |
| | | human | KDSTYSLSSTLTLSKADYEKHKVYACEVTHQGLSSPVTKSENRGEC |
| | | kappa | |
| | | background | |
| 13 | hH1 | Humanized | QVQLQESGPGLVKPSQTLSLTCTVSGYSITSDYAWNWIRQPPGKGLEWIGYIGYNG |
| | | 4E11 heavy | RTSYNPSLKSRVTISVDTSKNQFSLKLSSVTAADTAVYYCARLGRGFAYWGQGTLV |
| | | chain variant | TVSSASTKGPSVFPLAPCSRSTSESTAALGCLVKDYFPEPVTVSWNSGALTSGVHT |
| | | 1 on human | FPAVLOSSGLYSLSSVVTVPSSSLGTKTYTCNVDHKPSNTKVDKRVESKYGPPCPP |
| | | IgG4(S228P) | CPAPEFLGGPSVFLFPPKPKDTLMISRTPEVTCVVVDVSQEDPEVQFNWYVDGVEV |
| | | background | HNAKTKPREEQFNSTYRVVSVLTVLHQDWLNGKEYKCKVSNKGLPSSIEKTISKAK |
| | | | GQPREPQVYTLPPSQEEMTKNQVSLTCLVKGFYPSDIAVEWESNGQPENNYKTTPP |
| | | | VLDSDGSFFLYSRLTVDKSRWQEGNVFSCSVMHEALHNHYTQKSLSLSLG |
| 14 | hH2 | Humanized | QVQLQESGPGLVKPSQTLSLTCTVSGYSITSDYAWNWIRQPPGKGLEWIGYIGYNG |
| | | 4E11 heavy | RTSYNPSLKSRVTISRDTSKNQFSLKLSSVTAADTAVYYCARLGRGFAYWGQGTLV |
| | | chain variant | TVSSASTKGPSVFPLAPCSRSTSESTAALGCLVKDYFPEPVTVSWNSGALTSGVHT |
| | | 2 on human | FPAVLOSSGLYSLSSVVTVPSSSLGTKTYTCNVDHKPSNTKVDKRVESKYGPPCPP |
| | | IgG4(S228P) | CPAPEFLGGPSVFLFPPKPKDTLMISRTPEVTCVVVDVSQEDPEVQFNWYVDGVEV |
| | | background | HNAKTKPREEQFNSTYRVVSVLTVLHQDWLNGKEYKCKVSNKGLPSSIEKTISKAK |
| | | | GQPREPQVYTLPPSQEEMTKNQVSLTCLVKGFYPSDIAVEWESNGQPENNYKTTPP |
| | | | VLDSDGSFFLYSRLTVDKSRWQEGNVFSCSVMHEALHNHYTQKSLSLSLG |
| 15 | hH3 | Humanized | QVQLQESGPGLVKPSQTLSLTCTVSGYSITSDYAWNWIRQPPGKGLEWMGYIGYNG |
| | | 4E11 heavy | RTSYNPSLKSRITISRDTSKNQFSLKLSSVTAADTAVYYCARLGRGFAYWGQGTLV |
| | | chain variant | TVSSASTKGPSVFPLAPCSRSTSESTAALGCLVKDYFPEPVTVSWNSGALTSGVHT |
| | | 3 on human | FPAVLOSSGLYSLSSVVTVPSSSLGTKTYTCNVDHKPSNTKVDKRVESKYGPPCPP |
| | | IgG4(S228P) | CPAPEFLGGPSVFLFPPKPKDTLMISRTPEVTCVVVDVSQEDPEVQFNWYVDGVEV |
| | | background | HNAKTKPREEQFNSTYRVVSVLTVLHQDWLNGKEYKCKVSNKGLPSSIEKTISKAK |
| | | | GQPREPQVYTLPPSQEEMTKNQVSLTCLVKGFYPSDIAVEWESNGQPENNYKTTPP |
| | | | VLDSDGSFFLYSRLTVDKSRWQEGNVFSCSVMHEALHNHYTQKSLSLSLG |
| 16 | hL1 | Humanized | DIQMTQSPSSLSASVGDRVTITCHASQGINSNIGWYQQKPGKAPKLLIYHGTNLED |
| | | 4E11 light | GVPSRFSGSGSGTDYTLTISSLOPEDFATYYCVQYAQFPYTFGOGTKLEIKRTVAA |
| | | chain variant | PSVFIFPPSDEQLKSGTASVVCLLNNFYPREAKVOWKVDNALQSGNSQESVTEQDS |
| | | 1 on human | KDSTYSLSSTLTLSKADYEKHKVYACEVTHQGLSSPVTKSENRGEC |
| | | kappa | |
| | | background | |
| 17 | hL2 | Humanized | DIQMTQSPSSLSASVGDRVTITCHASQGINSNIGWLOQKPGKAPKGLIYHGTNLED |
| | | 4E11 light | GVPSRFSGSGSGTDYTLTISSLOPEDFATYYCVQYAQFPYTFGQGTKLEIKRTVAA |
| | | chain variant | PSVFIFPPSDEQLKSGTASVVCLLNNFYPREAKVOWKVDNALQSGNSQESVTEQDS |
| | | 2 on human | KDSTYSLSSTLTLSKADYEKHKVYACEVTHOGLSSPVTKSENRGEC |
| | | kappa | |
| | | background | |
| 18 | hL3 | Humanized | DIQMTQSPSSLSASVGDRVTITCHASQGINSNIGWLQQKPGKAFKGLIYHGTNLED |
| | | 4E11 light | GVPSRFSGSGSGTDYTLTISSLOPEDFATYYCVQYAQFPYTFGQGTKLEIKRTVAA |
| | | chain variant | PSVFIFPPSDEQLKSGTASVVCLLNNFYPREAKVQWKVDNALQSGNSQESVTEQDS |
| | | 3 on human | KDSTYSLSSTLTLSKADYEKHKVYACEVTHQGLSSPVTKSENRGEC |
| | | kappa | |
| | | background | |
| 19 | HSP | Signal | MDWTWRILFLVAAATGTHA |
| | | peptide on | |
| | | heavy chain | |
| | | (exemplary) | |
| 20 | LSP | Signal | MVLQTQVFISLLLWISGAYG |
| | | peptide on | |
| | | light chain | |
| | | (exemplary) | |
| 21 | hH1-SP | DNA | ATGGACTGGACCTGGAGGATCCTGTTCCTGGTGGCCGCCGCCACCGGCACCCACGCCCAG |
| | DNA | sequence of | GTGCAGCTGCAGGAGTCCGGCCCCGGCCTGGTGAAGCCCTCCCAGACCCTGTCCCTGACC |
| | | humanized | TGCACCGTGTCCGGCTACTCCATCACCTCCGACTACGCCTGGAACTGGATCAGGCAGCCC |
| | | 4E11 heavy | CCCGGCAAGGGCCTGGAGTGGATCGGCTACATCGGCTACAACGGCAGGACCTCCTACAAC |
| | | chain variant | CCCTCCCTGAAGTCCAGGGTGACCATCTCCGTGGACACCTCCAAGAACCAGTTCTCCCTG |
| | | 1 on human | AAGCTGTCCTCCGTGACCGCCGCCGACACCGCCGTGTACTACTGCGCCAGGCTGGGCAGG |
| | | IgG4(S228P) | GGCTTCGCCTACTGGGGCCAGGGCACCCTGGTGACCGTGTCCTCCGCCTCCACCAAGGGC |
| | | background | CCCTCCGTGTTCCCCCTGGCCCCCTGCTCCAGGTCCACCTCCGAGTCCACCGCCGCCCTG |
| | | including an | GGCTGCCTGGTGAAGGACTACTTCCCCGAGCCCGTGACCGTGTCCTGGAACTCCGGCGCC |
| | | exemplary | CTGACCTCCGGCGTGCACACCTTCCCCGCCGTGCTGCAGTCCTCCGGCCTGTACTCCCTG |
| | | signal | TCCTCCGTGGTGACCGTGCCCTCCTCCTCCCTGGGCACCAAGACCTACACCTGCAACGTG |
| | | peptide | GACCACAAGCCCTCCAACACCAAGGTGGACAAGAGGGTGGAGTCCAAGTACGGCCCCCCC |
| | | (underlined) | TGCCCCCCCTGCCCCGCCCCCGAGTTCCTGGGCGGCCCCTCCGTGTTCCTGTTCCCCCCC |
| | | | AAGCCCAAGGACACCCTGATGATCTCCAGGACCCCCGAGGTGACCTGCGTGGTGGTGGAC |
| | | | GTGTCCCAGGAGGACCCCGAGGTGCAGTTCAACTGGTACGTGGACGGCGTGGAGGTGCAC |
| | | | AACGCCAAGACCAAGCCCAGGGAGGAGCAGTTCAACTCCACCTACAGGGTGGTGTCCGTG |
| | | | CTGACCGTGCTGCACCAGGACTGGCTGAACGGCAAGGAGTACAAGTGCAAGGTGTCCAAC |
| | | | AAGGGCCTGCCCTCCTCCATCGAGAAGACCATCTCCAAGGCCAAGGGCCAGCCCAGGGAG |
| | | | CCCCAGGTGTACACCCTGCCCCCCTCCCAGGAGGAGATGACCAAGAACCAGGTGTCCCTG |
| | | | ACCTGCCTGGTGAAGGGCTTCTACCCCTCCGACATCGCCGTGGAGTGGGAGTCCAACGGC |
| | | | CAGCCCGAGAACAACTACAAGACCACCCCCCCCGTGCTGGACTCCGACGGCTCCTTCTTC |
| | | | CTGTACTCCAGGCTGACCGTGGACAAGTCCAGGTGGCAGGAGGGCAACGTGTTCTCCTGC |
| | | | TCCGTGATGCACGAGGCCCTGCACAACCACTACACCCAGAAGTCCCTGTCCCTGTCCCTG |
| | | | GGC |
| 22 | hH2-SP | DNA | ATGGACTGGACCTGGAGGATCCTGTTCCTGGTGGCCGCCGCCACCGGCACCCACGCCCAG |
| | DNA | sequence of | GTGCAGCTGCAGGAGTCCGGCCCCGGCCTGGTGAAGCCCTCCCAGACCCTGTCCCTGACC |
| | | humanized | TGCACCGTGTCCGGCTACTCCATCACCTCCGACTACGCCTGGAACTGGATCAGGCAGCCC |
| | | 4E11 heavy | CCCGGCAAGGGCCTGGAGTGGATCGGCTACATCGGCTACAACGGCAGGACCTCCTACAAC |
| | | chain variant | CCCTCCCTGAAGTCCAGGGTGACCATCTCCAGGGACACCTCCAAGAACCAGTTCTCCCTG |
| | | 2 on human | AAGCTGTCCTCCGTGACCGCCGCCGACACCGCCGTGTACTACTGCGCCAGGCTGGGCAGG |
| | | IgG4(S228P) | GGCTTCGCCTACTGGGGCCAGGGCACCCTGGTGACCGTGTCCTCCGCCTCCACCAAGGGC |
| | | background | CCCTCCGTGTTCCCCCTGGCCCCCTGCTCCAGGTCCACCTCCGAGTCCACCGCCGCCCTG |
| | | including an | GGCTGCCTGGTGAAGGACTACTTCCCCGAGCCCGTGACCGTGTCCTGGAACTCCGGCGCC |
| | | exemplary | CTGACCTCCGGCGTGCACACCTTCCCCGCCGTGCTGCAGTCCTCCGGCCTGTACTCCCTG |
| | | signal | TCCTCCGTGGTGACCGTGCCCTCCTCCTCCCTGGGCACCAAGACCTACACCTGCAACGTG |
| | | peptide | GACCACAAGCCCTCCAACACCAAGGTGGACAAGAGGGTGGAGTCCAAGTACGGCCCCCCC |
| | | (underlined) | TGCCCCCCCTGCCCCGCCCCCGAGTTCCTGGGCGGCCCCTCCGTGTTCCTGTTCCCCCCC |
| | | | AAGCCCAAGGACACCCTGATGATCTCCAGGACCCCCGAGGTGACCTGCGTGGTGGTGGAC |
| | | | GTGTCCCAGGAGGACCCCGAGGTGCAGTTCAACTGGTACGTGGACGGCGTGGAGGTGCAC |
| | | | AACGCCAAGACCAAGCCCAGGGAGGAGCAGTTCAACTCCACCTACAGGGTGGTGTCCGTG |
| | | | CTGACCGTGCTGCACCAGGACTGGCTGAACGGCAAGGAGTACAAGTGCAAGGTGTCCAAC |
| | | | AAGGGCCTGCCCTCCTCCATOGAGAAGACCATCTCCAAGGCCAAGGGCCAGCCCAGGGAG |
| | | | CCCCAGGTGTACACCCTGCCCCCCTCCCAGGAGGAGATGACCAAGAACCAGGTGTCCCTG |
| | | | ACCTGCCTGGTGAAGGGCTTCTACCCCTCCGACATCGCCGTGGAGTGGGAGTCCAACGGC |
| | | | CAGCCCGAGAACAACTACAAGACCACCCCCCCCGTGCTGGACTCCGACGGCTCCTTCTTC |
| | | | CTGTACTCCAGGCTGACCGTGGACAAGTCCAGGTGGCAGGAGGGCAACGTGTTCTCCTGC |
| | | | TCCGTGATGCACGAGGCCCTGCACAACCACTACACCCAGAAGTCCCTGTCCCTGTCCCTG |
| | | | GGC |
| 23 | hH3-SP | DNA | ATGGACTGGACCTGGAGGATCCTGTTCCTGGTGGCCGCCGCCACCGGCACCCACGCCCAG |
| | DNA | sequence of | GTGCAGCTGCAGGAGTCCGGCCCCGGCCTGGTGAAGCCCCCCAGACCCTGTCCCTGACC |
| | | humanized | TGCACCGTGTCCGGCTACTCCATCACCTCCGACTACGCCTGGAACTGGATCAGGCAGCCC |
| | | 4E11 heavy | CCCGGCAAGGGCCTGGAGTGGATGGGCTACATCGGCTACAACGGCAGGACCTCCTACAAC |
| | | chain variant | CCCTCCCTGAAGTCCAGGATCACCATCTCCAGGGACACCTCCAAGAACCAGTTCTCCCTG |
| | | 3 on human | AAGCTGTCCTCCGTGACCGCCGCCGACACCGCCGTGTACTACTGCGCCAGGCTGGGCAGG |
| | | IgG4(S228P) | GGCTTCGCCTACTGGGGCCAGGGCACCCTGGTGACCGTGTCCTCCGCCTCCACCAAGGGC |
| | | background | CCCTCCGTGTTCCCCCTGGCCCCCTGCTCCAGGTCCACCTCCGAGTCCACCGCCGCCCTG |
| | | including an | GGCTGCCTGGTGAAGGACTACTTCCCCGAGCCCGTGACCGTGTCCTGGAACTCCGGCGCC |
| | | exemplary | CTGACCTCCGGCGTGCACACCTTCCCCGCCGTGCTGCAGTCCTCCGGCCTGTACTCCCTG |
| | | signal | TCCTCCGTGGTGACCGTGCCCTCCTCCTCCCTGGGCACCAAGACCTACACCTGCAACGTG |
| | | peptide | GACCACAAGCCCTCCAACACCAAGGTGGACAAGAGGGGGAGTCCAAGTACGGCCCCCCC |
| | | (underlined) | TGCCCCCCCTGCCCCGCCCCCGAGTTCCTGGGCGGCCCCTCCGTGTTCCTGTTCCCCCCC |
| | | | AAGCCCAAGGACACCCTGATGATCTCCAGGACCCCCGAGGTGACCTGCGTGGTGGTGGAC |
| | | | GTGTCCCAGGAGGACCCCGAGGTGCAGTTCAACTGGTACGTGGACGGCGTGGAGGTGCAC |
| | | | AACGCCAAGACCAAGCCCAGGGAGGAGCAGTTCAACTCCACCTACAGGGTGGTGTCCGTG |
| | | | CTGACCGTGCTGCACCAGGACTGGCTGAACGGCAAGGAGTACAAGTGCAAGGTGTCCAAC |
| | | | AAGGGCCTGCCCTCCTCCATCGAGAAGACCATCTCCAAGGCCAAGGGCCAGCCCAGGGAG |
| | | | CCCCAGGTGTACACCCTGCCCCCCTOCCAGGAGGAGATGACCAAGAACCAGGTGTCCCTG |
| | | | ACCTGCCTGGTGAAGGGCTTCTACCCCTCCGACATCGCCGTGGAGTGGGAGTCCAACGGC |
| | | | CAGCCCGAGAACAACTACAAGACCACCCCCCCCGTGCTGGACTCCGACGGCTCCTTCTTC |
| | | | CTGTACTCCAGGCTGACCGTGGACAAGTCCAGGTGGCAGGAGGGCAACGTGTTCTCCTGC |
| | | | TCCGTGATGCACGAGGCCCTGCACAACCACTACACCCAGAAGTCCCTGTCCCTGTCCCTG |
| | | | GGC |
| 24 | hL1-SP | DNA | ATGGTGCTGCAGACCCAGGTGTTCATCTCCCTGCTGCTGTGGATCTCCGGCGCCTACGGC |
| | DNA | sequence of | GACATCCAGATGACCCAGTCCCCCTCCTCCCTGTCCGCCTCCGTGGGCGACAGGGTGACC |
| | | humanized | ATCACCTGCCACGCCTCCCAGGGCATCAACTCCAACATCGGCTGGTACCAGCAGAAGCCC |
| | | 4E11 light | GGCAAGGCCCCCAAGCTGCTGATCTACCACGGCACCAACCTGGAGGACGGCGTGCCCTCC |
| | | chain variant | AGGTTCTCCGGCTCCGGCTCCGGCACCGACTACACCCTGACCATCTCCTCCCTGCAGCCC |
| | | 1 on human | GAGGACTTCGCCACCTACTACTGCGTGCAGTACGCCCAGTTCCCCTACACCTTCGGCCAG |
| | | kappa | GGCACCAAGCTGGAGATCAAGAGGACCGTGGCCGCCCCCTCCGTGTTCATCTTCCCCCCC |
| | | background | TCCGACGAGCAGCTGAAGTCCGGCACCGCCTCCGTGGTGTGCCTGCTGAACAACTTCTAC |
| | | including an | CCCAGGGAGGCCAAGGTGCAGTGGAAGGTGGACAACGCCCTGCAGTCCGGCAACTCCCAG |
| | | exemplary | GAGTCCGTGACCGAGCAGGACTCCAAGGACTCCACCTACTCCCTGTCCTCCACCCTGACC |
| | | signal | CTGTCCAAGGCCGACTACGAGAAGCACAAGGTGTACGCCTGCGAGGTGACCCACCAGGGC |
| | | peptide | CTGTCCTCCCCCGTGACCAAGTCCTTCAACAGGGGCGAGTGC |
| | | (underlined) | |
| 25 | hL2-SP | DNA | ATGGTGCTGCAGACCCAGGTGTTCATCTCCCTGCTGCTGTGGATCTCCGGCGCCTACGGC |
| | DNA | sequence of | GACATCCAGATGACCCAGTCCCCCTCCTCCCTGTCCGCCTCCGTGGGCGACAGGGTGACC |
| | | humanized | ATCACCTGCCACGCCTCCCAGGGCATCAACTCCAACATCGGCTGGOTGCAGCAGAAGCCC |
| | | 4E11 light | GGCAAGGCCCCCAAGGGCCTGATCTACCACGGCACCAACCTGGAGGACGGCGTGCCCTCC |
| | | chain variant | AGGTTCTCCGGCTCCGGCTCCGGCACCGACTACACCCTGACCATCTCCTCCCTGCAGCCC |
| | | 2 on human | GAGGACTTCGCCACCTACTACTGCGTGCAGTACGCCCAGTTCCCCTACACCTTCGGCCAG |
| | | kappa | GGCACCAAGCTGGAGATCAAGAGGACCGTGGCCGCCCCCTCCGTGTTCATCTTCCCCCCC |
| | | background | TCCGACGAGCAGCTGAAGTCCGGCACCGCCTCCGTGGTGTGCCTGCTGAACAACTTCTAC |
| | | including an | CCCAGGGAGGCCAAGGTGCAGTGGAAGGTGGACAACGCCCTGCAGTCCGGCAACTCCCAG |
| | | exemplary | GAGTCCGTGACCGAGCAGGACTCCAAGGACTCCACCTACTCCCTGTCCTCCACCCTGACC |
| | | signal | CTGTCCAAGGCCGACTACGAGAAGCACAAGGTGTACGCCTGCGAGGTGACCCACCAGGGC |
| | | peptide | CTGTCCTCCCCCGTGACCAAGTCCTTCAACAGGGGCGAGTGC |
| | | (underlined) | |
| 26 | hL3-SP | DNA | ATGGTGCTGCAGACCCAGGTGTTCATCTCCCTGCTGCTGTGGATCTCCGGCGCCTACGGC |
| | DNA | sequence of | GACATCCAGATGACCCAGTCCCCCTCCTCCCTGTCCGCCTCCGTGGGCGACAGGGTGACC |
| | | humanized | ATCACCTGCCACGCCTCCCAGGGCATCAACTCCAACATCGGCTGGOTGCAGCAGAAGCCC |
| | | 4E11 light | GGCAAGGCCTTCAAGGGCCTGATCTACCACGGCACCAACCTGGAGGACGGCGTGCCCTCC |
| | | chain variant | AGGTTCTCCGGCTCCGGCTCCGGCACCGACTACACCCTGACCATCTCCTCCCTGCAGCCC |
| | | 3 on human | GAGGACTTCGCCACCTACTACTGCGTGCAGTACGCCCAGTTCCCCTACACCTTCGGCCAG |
| | | kappa | GGCACCAAGCTGGAGATCAAGAGGACCGTGGCCGCCCCCTCCGTGTTCATCTTCCCCCCC |
| | | background | TCCGACGAGCAGCTGAAGTCCGGCACCGCCTCCGTGGTGTGCCTGCTGAACAACTTCTAC |
| | | including an | CCCAGGGAGGCCAAGGTGCAGTGGAAGGTGGACAACGCCCTGCAGTCCGGCAACTCCCAG |
| | | exemplary | GAGTCCGTGACCGAGCAGGACTCCAAGGACTCCACCTACTCCCTGTCCTCCACCCTGACC |
| | | signal | CTGTCCAAGGCCGACTACGAGAAGCACAAGGTGTACGCCTGCGAGGTGACCCACCAGGGC |
| | | peptide | CTGTCCTCCCCCGTGACCAAGTCCTTCAACAGGGGCGAGTGC |
| | | (underlined) | |

1. An antigen-binding agent, antigen-binding domain, antibody or antigen-binding fragment thereof that specifically binds to epidermal growth factor receptor variant III (EGFRvIII), wherein the antigen-binding agent, antigen-binding domain, antibody or antigen-binding fragment comprises:
a. a heavy chain variable region comprising the amino acid sequence QVQLQESGPGLVKPSQTLSLTCTVSGYSITSDYAWNWIRQPPGKGLEWX₁GYI GYNGRTSYNPSLKSRX₂TISX₃DTSKNQFSLKLSSVTAADTAVYYCARLGRGFAY WGQGTLVTVSS (SEQ ID NO:3), where X1=I or M, X2=V or I, and X3=V or R; and,
b. a light chain variable region comprising the amino acid sequence DIQMTQSPSSLSASVGDRVTITCHASQGINSNIGWX₄QQKPGKAX₅KX₆LIYHGT NLEDGVPSRFSGSGSGTDYTLTISSLOPEDFATYYCVQYAQFPYTFGQGTKLEI K (SEQ ID NO:4), where X₄=Y or L, X₅=P or F, and X₆=L or G.

2. The antigen-binding agent, antigen-binding domain, antibody or antigen-binding fragment of claim 1, wherein:
a. the heavy chain variable sequence is selected from any one of SEQ ID NO: 5, SEQ ID NO: 6 and SEQ ID NO 7; and,
b. the light chain variable sequence is selected from any one of SEQ ID NO: 8, SEQ ID NO: 9 and SEQ ID NO: 10.

3. The antigen-binding agent, antigen-binding domain, antibody or antigen-binding fragment of claim 1 or 2, wherein the antigen-binding agent, antigen-binding domain, antibody or antigen-binding fragment thereof is a chimeric antigen receptor, a bi-specific T-cell engager, a bispecific killer cell engager, a trispecific killer cell engager or any immunotherapeutic compound.

4. The antigen-binding agent, antigen-binding domain, antibody or antigen-binding fragment of claim 3, wherein the antibody is a monoclonal antibody, a polyclonal antibody, a humanized antibody, a chimeric antibody, a human antibody, a single chain antibody, or a multispecific antibody.

5. The antigen-binding agent, antigen-binding domain, antibody or antigen-binding fragment of claim 4, wherein the antibody or antigen-binding fragment thereof comprises a human IgG constant region.

6. The antigen-binding agent, antigen-binding domain, antibody or antigen-binding fragment of claim 5, wherein the antibody or antigen-binding fragment thereof comprises a human IgG4 constant region.

7. The antigen-binding agent, antigen-binding domain, antibody or antigen-binding fragment of claim 4, wherein the antibody or antigen-binding fragment thereof comprises a human IgG4 constant region bearing the S228P mutation.

8. An antigen-binding agent, antigen-binding domain, antibody or antigen-binding fragment of claim 7 which specifically binds to EGFRvIII wherein the antigen-binding agent, antigen-binding domain, antibody or antigen-binding fragment comprises a heavy chain sequence and a light chain sequence, wherein:
a. the heavy chain sequence is SEQ ID NO: 13, SEQ ID NO: 14 or SEQ ID NO: 15; and,
b. the light chain sequence is SEQ ID NO: 16, SEQ ID NO: 17 or SEQ ID NO: 18.

9. The antigen-binding agent, antigen-binding domain, antibody or antigen-binding fragment of any one of claims 3 to 8, wherein the antigen-binding agent, antigen-binding domain, antibody or antigen-binding fragment comprises a scFv, a Fab, a Fab′ or a (Fab′)₂.

10. The antigen-binding agent, antigen-binding domain, antibody or antigen-binding fragment of any one of claims 3 to 9, wherein the antigen-binding agent, antigen-binding domain, antibody or antigen-binding fragment is linked to a cargo molecule.

11. The antigen-binding agent, antigen-binding domain, antibody or antigen-binding fragment of claim 10, wherein the cargo molecule comprises a therapeutic moiety.

12. The antigen-binding agent, antigen-binding domain, antibody or antigen-binding fragment of claim 11, wherein the therapeutic moiety comprises a cytotoxic agent, a cytostatic agent, an anti-cancer agent or a radiotherapeutic.

13. The antigen-binding agent, antigen-binding domain, antibody or antigen-binding fragment of claim 11, wherein the antigen-binding agent, antigen-binding domain, antibody or antigen-binding fragment is conjugated to a detectable moiety.

14. A pharmaceutical composition comprising the antigen-binding agent, antigen-binding domain, antibody or antigen-binding fragment of any one of claims 1 to 13 and a pharmaceutically acceptable carrier, diluent or excipient.

15. A nucleic acid molecule encoding a heavy chain variable region and/or a light chain variable region of the antigen-binding agent, antigen-binding domain, antibody or antigen-binding fragment of any one of claims 1 to 9.

16. A kit comprising at least one of the antigen-binding agent, antigen-binding domain, antibody or antigen-binding fragment of any one of claims 1 to 15.

17. A vector or set of vectors comprising a nucleic acid sequence encoding a heavy chain variable region and a light chain variable region of the antigen-binding agent, antigen-binding domain, antibody or antigen-binding fragment of any one of claims 1 to 11.

18. An isolated cell comprising the vector or set of vectors of claim 17.

19. The isolated cells of claim 18, wherein said cell is capable of expressing, assembling and/or secreting an antigen-binding agent, antigen-binding domain, antibody or antigen-binding fragment thereof.

20. A kit comprising a first vial comprising a nucleotide or vector encoding the light chain of the antigen-binding agent, antigen-binding domain, antibody or antigen-binding fragment of any one of claims 1 to 9 and a second vial comprising a nucleotide or vector encoding the heavy chain of the antigen-binding agent, antigen-binding domain, antibody or antigen-binding fragment of any one of claims 1 to 9.

21. A method of treating cancer comprising cells expressing EGFRvIII, the method comprising administering the antigen-binding agent, antigen-binding domain, antibody or antigen-binding fragment of any one of claims 1 to 14 to a subject in need.

22. The method of claim 21, wherein the antigen-binding agent, antigen-binding domain, antibody or antigen-binding fragment is used in combination with a chemotherapeutic.

23. The method of any one of claim 21 or 22, wherein the subject in need has or is suspected of having gliobastoma multiforme.

24. The method of any one of claim 21 or 22, wherein the subject in need has or is suspected of having a carcinoma.

25. The method of claim 24, wherein the carcinoma comprises breast carcinoma or HNSCC.

26. A method of detecting EGFRvIII, the method comprising contacting a sample comprising or suspected of comprising EGFRvIII with the antigen-binding agent, antigen-binding domain, antibody or antigen-binding fragment of any one of claims 1 to 14.

27. A method of making the antigen-binding agent, antigen-binding domain, antibody or antigen-binding fragment of any one of claims 1 to 9, comprising culturing a cell comprising nucleic acids encoding said antigen-binding agent, antigen-binding domain, antibody or antigen-binding fragment so that the antigen-binding agent, antigen-binding domain, antibody or antigen-binding fragment is produced.

28. The method of claim 27, further comprising conjugating the antigen-binding agent, antigen-binding domain, antibody or antigen-binding fragment thereof with a cargo molecule.

29. The method of claim 28, wherein the cargo molecule comprises a therapeutic moiety.

30. The method of claim 29, wherein the cargo molecule comprises a detectable moiety.

31. A method of treating subject having a cancer associated with EGFRvIII expression, the method comprising administering cells expressing the antigen-binding agent, antigen-binding domain, antibody or antigen-binding fragment of any one of claims 1 to 3, wherein the antigen-binding agent, antigen-binding domain, antibody or antigen-binding fragment is a chimeric antigen receptor, a bi-specific T-cell engager, a bispecific killer cell engager or a trispecific killer cell engage or an antibody drug conjugate.

32. The method of claim 31, wherein the subject in need has or is suspected of having glioma.

33. The method of claim 32, wherein the glioma is gliobastoma multiforme.

34. The method of claim 31, wherein the subject in need has or is suspected of having a carcinoma.

35. The method of claim 34, wherein the carcinoma comprises breast carcinoma, oral carcinoma or HNSCC.

36. The method of any one of claims 31 to 35, wherein the cells are T-cells.

37. The method of any one of claims 31 to 36, wherein the cells are NK cells.

38. The method of any one of claims 31 to 47, wherein the cells are immune cells autologous to the subject.

39. An isolated cell population engineered to express the antigen-binding agent, antigen-binding domain, antibody or antigen-binding fragment of any one of claims 1 to 3.

40. The isolated cell population of claim 39, wherein the isolated cell population is of human origin.

41. The isolated cell population of claim 39 or 40, wherein the isolated cell population comprises T cells, Natural Killer (NK) cells, cytotoxic T cells, regulatory T cells, and combinations thereof.

42. The isolated cell population of claim 41, wherein the isolated cell population comprises T cells.

43. The isolated cell population of claim 42, wherein the T-cells comprise CD4+ T-cells, CD8+ T-cells or a combination thereof.

44. The isolated cell population of claim 41, wherein the isolated cell population comprises NK cells.

45. The isolated cell population of any one of claims 39 to 44, wherein the isolated cell population is engineered to express another chimeric antigen receptor having affinity for another antigen of the same target or of a different target.

46. The isolated cell population of any one of claims 39 to 44, wherein the isolated cell population comprises a host's immune cells.

47. A pharmaceutical composition comprising the isolated cell population of any one of claims 39 to 46 and a pharmaceutically acceptable buffer or excipient.
